(19) United States
(12) Patent Application Publication
(10) Pub. No.: US 2026/0232643 A1
(43) Pub. Date: Aug. 13, 2026

(54) METHODS OF TREATING SOLID TUMORS WITH MITOGEN ACTIVATED PROTEIN KINASE (MAPK) PATHWAY ALTERATIONS

(71) Applicant: Jazz Pharmaceuticals Ireland Limited, Dublin (IE)

(72) Inventors: Ronald CHEUNG, Redwood City, CA (US); Robin C. HUMPHREYS, Dillon, CO (US); Svetlana MARKOVA, Sunnyvale, CA (US)

(21) Appl. No.: 19/156,994

(22) PCT Filed: Feb. 20, 2024
(86) PCT No.: PCT/US2024/016468

§ 371 Date: Aug. 15, 2025

Related U.S. Application Data
(60) Provisional application No. 63/485,621, filed on Feb. 17, 2023.

(51) Int. Cl. A61K 31/4375; A61P 35/00

(52) U.S. Cl. CPC A61K 31/4375; A61P 35/00

(57) ABSTRACT

The present disclosure relates to methods of treating a solid tumor by administering a therapeutically effective amount of a mitogen activated protein kinase (MAPK) inhibitor having a structure, Compound: (I), or a pharmaceutically acceptable salt thereof.

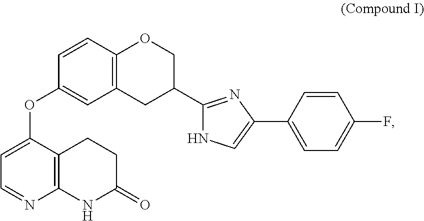 [SMILES: O=C1CCc2c(Oc3ccc4c(c3)CC(c3nc(-c5ccc(F)cc5)c[nH]3)CO4)ccnc2N1]

### CROSS-REFERENCE TO RELATED APPLICATION

This application claims the benefit of priority to U.S. Provisional Application No. 63/485,621, filed Feb. 17, 2023, which is hereby incorporated by reference in its entirety for all purposes.

### BACKGROUND

Mutations leading to uncontrolled signaling via the RAS-RAF-MAPK pathway are seen in more than one third of all cancers. RAF kinases (A-RAF, B-RAF and C-RAF) are an integral part of this pathway, with B-RAF mutations commonly seen in the clinic. Interestingly, certain types of B-RAF mutation-associated cancers are insensitive to the FDA-approved B-RAF selective drugs, due to the functions of other RAF family members. In particular, B-RAF selective monotherapies fail to show clinical benefit against atypical B-RAF (non-V600E), other RAF and RAS driven tumors.

There is a need in the art to provide alternative and improved methods for treating solid tumors that are insensitive to currently available B-RAF selective drugs.

The present application addresses these needs.

### SUMMARY

The present disclosure provides methods of treating cancer in a subject in need thereof. In particular, the present disclosure provides methods of treating a solid tumor by administering a pan-RAF inhibitor to a subject having a solid tumor harboring one or more MAPK pathway alterations.

In one aspect, the present disclosure provides a method of treating cancer, comprising administering about 5 mg to about 1500 mg of Compound I:

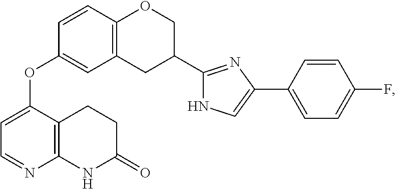 [SMILES: O=C1CCc2c(Oc3ccc4c(c3)CC(c3nc(-c5ccc(F)cc5)c[nH]3)CO4)ccnc2N1]

or an equivalent dose of a pharmaceutically acceptable salt thereof, to a subject in need thereof. In embodiments, about 5 mg to about 500 mg of Compound I or an equivalent dose of a pharmaceutically acceptable salt thereof, is administered to the subject.

In embodiments, Compound I is:

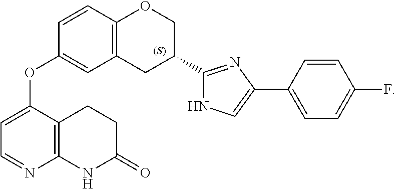

In embodiments, the Compound I of the present disclosure has an enantiomeric excess of at least 85%, at least 90%, at least 95%, or at least 99%.

In embodiments, about 5 mg, about 10 mg, about 20 mg, about 25 mg, about 30 mg, about 40 mg, about 50 mg, about 60 mg, about 75 mg, about 80 mg, about 100 mg, about 150 mg, about 175 mg, about 200 mg, about 225 mg, about 275 mg, about 325 mg, about 375 mg, about 425 mg, about 475 mg, about 500 mg, about 800 mg, about 1000 mg, about 1200 mg, or about 1500 mg of Compound I, or an equivalent dose of a pharmaceutically acceptable salt thereof, is administered. In embodiments, about 20 mg to about 1000 mg or about 20 mg to about 500 mg of Compound I, or an equivalent dose of a pharmaceutically acceptable salt thereof, is administered per day. In embodiments, about 40 mg to about 1000 mg or about 40 mg to about 500 mg of Compound I, or an equivalent dose of a pharmaceutically acceptable salt thereof, is administered per day. In embodiments, about 5 mg, about 10 mg, about 20 mg, about 25 mg, about 30 mg, about 40 mg, about 50 mg, about 60 mg, about 75 mg, about 80 mg, about 100 mg, about 150 mg, about 175 mg, about 200 mg, about 225 mg, about 275 mg, about 325 mg, about 375 mg, about 425 mg, about 475 mg, about 500 mg, of Compound I, or an equivalent dose of a pharmaceutically acceptable salt thereof, is administered. In embodiments, about 1000 mg or about 500 mg of Compound I, or an equivalent dose of a pharmaceutically acceptable salt thereof, is administered per day.

In embodiments, Compound I, or an equivalent dose of a pharmaceutically acceptable salt thereof, is administered once a day or twice a day. In embodiments, about 20 mg of Compound I, or an equivalent dose of a pharmaceutically acceptable salt thereof, is administered twice a day. In embodiments, about 30 mg of Compound I, or an equivalent dose of a pharmaceutically acceptable salt thereof, is administered twice a day. In embodiments, about 50 mg of Compound I, or an equivalent dose of a pharmaceutically acceptable salt thereof, is administered once a day. In embodiments, about 50 mg of Compound I, or an equivalent dose of a pharmaceutically acceptable salt thereof, is administered twice a day. In embodiments, about 75 mg of Compound I, or an equivalent dose of a pharmaceutically acceptable salt thereof, is administered twice a day. In embodiments, about 100 mg of Compound I, or an equivalent dose of a pharmaceutically acceptable salt thereof, is administered once a day.

In embodiments, about 250 mg of Compound I, or an equivalent dose of a pharmaceutically acceptable salt thereof, is administered twice a day. In embodiments, 500 mg of Compound I or an equivalent dose of a pharmaceutically acceptable salt thereof, is administered twice a day.

In embodiments, Compound I, or an equivalent dose of a pharmaceutically acceptable salt thereof, is orally administered in a pharmaceutical formulation. In embodiments, the pharmaceutical formulation is a tablet or a capsule. In embodiments, the pharmaceutical formulation is a capsule. In embodiments, the pharmaceutical formulation comprises about 5 mg to about 50 mg of Compound I, or an equivalent dose of a pharmaceutically acceptable salt thereof. In embodiments, the pharmaceutical formulation comprises about 10 mg or about 25 mg of Compound I, or an equivalent dose of a pharmaceutically acceptable salt thereof.

In embodiments, the cancer is advanced or metastatic cancer. In embodiments, the cancer is Barret's adenocarcinoma; biliary tract carcinomas; breast cancer; cervical cancer; cholangiocarcinoma; central nervous system tumors; primary CNS tumors; glioblastomas; astrocytomas; glioblastoma multiforme; ependymomas; secondary CNS tumors (metastases to the central nervous system of tumors originating outside of the central nervous system); brain tumors; brain metastases; colorectal cancer; large intestinal colon carcinoma; gastric cancer; carcinoma of the head and neck; squamous cell carcinoma of the head and neck; acute lymphoblastic leukemia; acute myelogenous leukemia (AML); myelodysplastic syndromes; chronic myelogenous leukemia; Hodgkin's lymphoma; non-Hodgkin's lymphoma; megakaryoblastic leukemia; multiple myeloma; erythroleukemia; hepatocellular carcinoma; lung cancer; small cell lung cancer; non-small cell lung cancer; ovarian cancer; endometrial cancer; pancreatic cancer; pituitary adenoma; prostate cancer; renal cancer; melanoma; neurofibromatosis type 1 (NF1); neurofibromatosis type 2 (NF2); esophageal cancer; bladder cancer; mesothelioma; or thyroid cancer. In embodiments, the cancer is non-small cell lung cancer (NSCLC), colorectal cancer, melanoma, or pancreatic cancer. In some embodiments, melanoma is metastatic melanoma.

In embodiments, the cancer comprises a solid tumor harboring one or more alterations in the mitogen activated protein kinase (MAPK) pathway. In embodiments, the one or more alterations is a KRAS mutation, a NRAS mutation, a HRAS mutation, a BRAF mutation, a BRAF fusion, a BRAF translocation, an ARAF mutation, RAF mutation, an increased receptor tyrosine kinase (RTK) signaling, RAS gene amplification, RAF gene amplification, or a combination thereof. In embodiments, the KRAS mutation is G12C, G12D, G12R, G12V, G12W, G13C, G13D, G13R, G13V, G13W, Q61R, Q61K, Q61L, Q61H, Q61P, A146P, A146T, A146V, A59G, A59T, R68S, H95D, H95Q, H95R, Y96C, or a combination thereof. In embodiments, the NRAS mutation comprises Q61R, Q61K, Q61L, Q61H, Q61P, G12S, G12D, G12C, G12V, G12A, G12R, G13V, G13R, G13D, G13C, or a combination thereof. In embodiments, the HRAS mutation comprises Q61R, Q61K, Q61L, G12S, G12D, G12C, G12V, G12A, G12R, G13V, G13R, G13D, G13C, or a combination thereof. In embodiments, the ARAF mutation comprises G331C, A225V, F351L, P539H, Q347_A348del, N217I, G322S, A451T, R255Gfs(Ter)37, S214(Ter), P8Lfs(Ter)26, G387N/D/R/S, P462L, G377R, or a combination thereof. In embodiments, the BRAF mutation comprises Class 1 BRAF mutation, Class 2 BRAF mutation, Class 3 BRAF mutation, or a combination thereof. In embodiments, the BRAF mutation comprises Class 2 or 3 BRAF mutation. In embodiments, the BRAF mutation comprises V600E, V600M, V600G, V600K, V600R, V600D, G469A, G469V, G469R, K601E, K601N, K601T, L597Q, L597V, G464V, G464E, D594N, D594G, D594A, D594H, G466A, G466V, G466E, N581S, N581I, G469E, S467L, G596D, G596R, F595L, V459L, D287H or a combination thereof. In embodiments, the RAF mutation comprises S257L/W, S259F, R391S/W, or a combination thereof. In embodiments, the one or more alterations comprises NRAS Q61K, KRAS G12C, KRAS G13D, KRAS Q61K, or a combination thereof.

In another aspect, the present disclosure provides a method of treating cancer harboring one or more alterations in the mitogen activated protein kinase (MAPK) pathway in a subject, comprising: detecting the one or more alterations in the MAPK pathway; and then administering about 5 mg to about 1500 mg of Compound I:

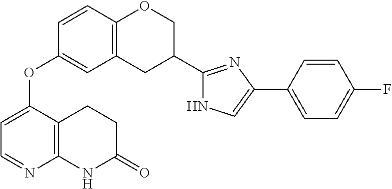 [SMILES: O=C1CCc2c(Oc3ccc4c(c3)CC(c3nc(-c5ccc(F)cc5)c[nH]3)CO4)ccnc2N1]

or an equivalent dose of a pharmaceutically acceptable salt thereof, to the subject. In embodiments, about 5 mg to about 500 mg of Compound I or an equivalent dose of a pharmaceutically acceptable salt thereof, is administered to the subject.

In embodiments, Compound I is

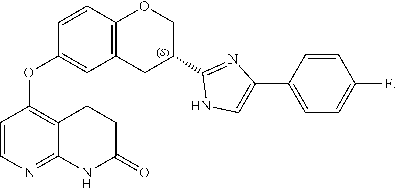

In embodiments, the Compound I of the present disclosure has an enantiomeric excess of at least 85%, at least 90%, at least 95%, or at least 99%.

In embodiments, the method of the present disclosure provides treatment that reduces the tumor size compared to the tumor size prior to administration. In embodiments, the treatment increases overall response rate (ORR) compared to the ORR of an otherwise similar patient with cancer that is not treated with Compound I. In embodiments, the treatment increases progression free survival (PFS) compared to the PFS of an otherwise similar patient with cancer that is not treated with Compound I. In embodiments, the treatment increases overall survival (OS) compared to the OS of an otherwise similar patient with cancer that is not treated with Compound I.

In embodiments, the treatment reduces tumor size by at least about 5%, about 10%, about 20%, about 30%, about 40%, about 50% about 60%, about 70%, about 80%, about 90%, about 95%, or about 100%, compared to the tumor size prior to administration. In embodiments, the treatment increases ORR by at least about 5%. In embodiments, the treatment increases ORR by 10% to about 100%. In embodiments, the treatment increases PFS by at least about 3 months. In embodiments, the treating increases PFS by at least about 6 months, 12 months, at least about 18 months, or at least about 24 months. In embodiments, the treatment increases OS by at least about 3 months.

In embodiments, the present disclosure provides a method of treating cancer expressing one or more ARAF mutants in a subject in need thereof, comprising administering about 5 mg to about 1500 mg of Compound I or an equivalent dose of a pharmaceutically acceptable salt thereof, to the subject in need thereof. In embodiments, about 5 mg to about 500 mg of Compound I or an equivalent dose of a pharmaceutically acceptable salt thereof, is administered to the subject.

In embodiments, the present disclosure provides a method of treating belvarafenib resistant cancer in a patient in need thereof, comprising administering about 5 mg to about 1500 mg of Compound I or an equivalent dose of a pharmaceutically acceptable salt thereof, to the subject in need thereof. In embodiments, about 5 mg to about 500 mg of Compound I or an equivalent dose of a pharmaceutically acceptable salt thereof, is administered to the subject.

### BRIEF DESCRIPTION OF THE DRAWINGS

FIGS. 1A-E show changes in tumor size (mm³) following administration of Compound I (Comp 1) at 10 mg/kg (BID), 30 mg/kg (BID), 30 mg/kg (QD), and 100 mg/kg (QD) to xenograft mice having NRAS, KRAS, and BRAF Class 2/3 mutation driven colorectal cancer (CRC) (FIG. 1A), non-small cell lung cancer (NSCLC) (FIG. 1B), melanoma (FIG. 1C), or ovarian cancer (FIG. 1D), as described in Example 3. Vehicle was administered BID as a control. The changes in tumor volume observed in ovarian cancer xenograft mice of FIG. 1D following 10 mg/kg (BID), 30 mg/kg (BID), and 100 mg/kg (QD) Compound I administration are shown in FIG. 1E. P<0.001 denoted by “***” and P<0.0001 denoted by “****” versus vehicle control.

FIG. 2 shows a schematic overview of part A and part B of the Phase 1 study described in Example 4. Abbreviations: MTD=maximum tolerated dose; NSCLC=non-small cell lung cancer; RP2D=recommended phase 2 dose.

FIG. 3 shows Positron Emission Tomography-Computed Tomography (PET/CT) images of a cancer patient before and after Compound I treatment, as described in Example 5.

### DETAILED DESCRIPTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

### Definitions

While the following terms are believed to be well understood by one of ordinary skill in the art, the following definitions are set forth to facilitate explanation of the presently disclosed subject matter.

As used in the specification and claims, the singular form “a”, “an” and “the” includes plural references unless the context clearly dictates otherwise.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range. As used herein, the term “substantially” means greater than 85% (i.e., greater than 85, 86, 87, 88, 89, 90, 91, 92, 93, 94, 95, 96, 97, 98, 99, or 100%).

As used herein, Compound I is 5-((3-(4-(4-fluorophenyl)-1H-imidazol-2-yl) chroman-6-yl)oxy)-3,4-dihydro-1,8-naphthyridin-2 (1H)-one having the structure shown below.

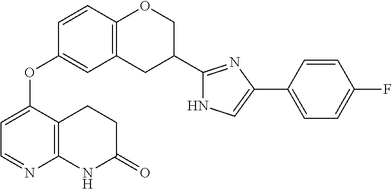 [SMILES: O=C1CCc2c(Oc3ccc4c(c3)CC(c3nc(-c5ccc(F)cc5)c[nH]3)CO4)ccnc2N1]

All references to Compound I herein include all stereoisomers and tautomers thereof. In embodiments, Compound I includes stereoisomers thereof. In embodiments, Compound I is (S)-5-((3-(4-(4-fluorophenyl)-1H-imidazol-2-yl) chroman-6-yl)oxy)-3,4-dihydro-1,8-naphthyridin-2 (1H)-one having the structure shown below.

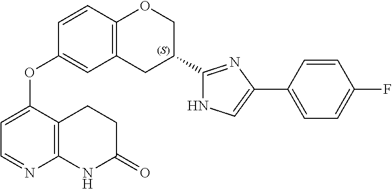

In embodiments, the Compound I of the present disclosure has an enantiomeric excess of at least 85%, at least 90%, at least 95%, or at least 99%.

Compound I can be prepared according to the methods disclosed in WIPO Patent Appln Pub. No. 2022/023450, which is incorporated herein for its entirety. All references to Compound I here also include pharmaceutically acceptable salt thereof. In embodiments, Compound I is a pan-RAF inhibitor.

The term “treating” means one or more of relieving, alleviating, delaying, reducing, reversing, improving, or managing at least one symptom of a condition in a subject. The term “treating” may also mean one or more of arresting, delaying the onset (i.e., the period prior to clinical manifestation of the condition) or reducing the risk of developing or worsening a condition.

As used herein, a daily dosage can be achieved by a single administration of the targeted dosage amount or multiple administrations of smaller dosage amount(s) over a 24-hour period. For example, a 150 mg daily dosage can be achieved by a single administration of 150 mg of the therapeutic agent per day, two administrations of 75 mg of the therapeutic agent per day, or three administrations of 50 mg of the therapeutic agent per day, or the like.

The term “therapeutically effective amount” and the like, as used herein, indicate an amount necessary to administer to a patient, or to a cell, tissue, or organ of a patient, to achieve a therapeutic effect, such as an ameliorating or a curative effect in treating cancer. The therapeutically effective amount is sufficient to elicit the biological or medical response of a cell, tissue, system, animal, or human that is being sought by a researcher, veterinarian, medical doctor, or clinician.

In certain embodiments, the pharmaceutically acceptable form is a pharmaceutically acceptable salt or a stereoisomer. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In embodiments, organic acids from which salts may be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Pharmaceutically acceptable salts derived from appropriate bases include alkali metal, alkaline earth metal, ammonium and N(C₁₋₄ alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate. Organic bases from which salts may be derived include, for example, primary, secondary, and tertiary amines, substituted amines including naturally occurring substituted amines, cyclic amines, basic ion exchange resins, and the like, such as isopropylamine, trimethylamine, diethylamine, triethylamine, tripropylamine, and ethanolamine. In embodiments, the pharmaceutically acceptable base addition salt is chosen from ammonium, potassium, sodium, calcium, and magnesium salts.

The term “equivalent dose” refers to, when comparing Compound I and a salt form, the dose containing the same amount of Compound I within about 5% (by weight %).

The terms “diagnose”, “diagnosis”, and “diagnosing”, as used herein, refer to identifying, assessing, evaluating, or classifying the presence or characteristics of a pathological condition, for example, diagnosing whether or not a subject referred to according to the method of the present disclosure has one or more alterations in the MAPK signaling pathway.

The term “subject” or “patient” refers to a mammal. In embodiments, the subject is a human.

The following description includes information that may be useful in understanding the present invention. It is not an admission that any of the information provided herein is prior art or relevant to the presently claimed inventions, or that any publication specifically or implicitly referenced is prior art.

### Methods of the Present Disclosure

Abnormal activation of the MAPK pathway can drive human cancers through the promotion of cell proliferation and growth. Mutations in genes involved in this pathway, including RAS and RAF, are common in human cancer such as melanoma, NSCLC, colorectal cancer, pancreatic cancer, especially in solid tumors. The most common mutations in the MAPK pathway occur in the RAS protein kinases (KRAS, NRAS, and HRAS), which lead to constitutive downstream activation of the effector RAF proteins (ARAF, BRAF, and CRAF). Although there have been RAF selective inhibitors developed for cancer treatment (e.g., vemurafenib, dabrafenib, and encorafenib), the clinical efficacy of these inhibitors is limited by the development of drug resistance in various steps in the MAPK signaling pathway, and limited or no activity against ARAF and ARAF mutants. These limitations of BRAF-selective inhibitors highlight the need for agents that can inhibit the activity of all RAF protein alterations, including the 3 classes of RAF mutants, namely BRAF V600E (Class I), BRA-CRAF dimer (Class II), and RAF dimer dependent mutants.

Applicant has surprisingly discovered that Compound I is a pan-RAF inhibitor. Applicant's experimental results demonstrated that Compound I is significantly more potent in inhibiting the kinase activity of all three RAF isoforms than known pan-RAF inhibitors. One of the most studied pan-RAF inhibitors is belvarafenib, which is a type II pan-RAF inhibitor currently undergoing clinical studies for treating BRAF V600E and NRAS-mutant melanoma. Although belvarafenib showed preclinical efficacy in decreasing tumor growth, it also showed acquired resistance in certain cancers. Through a combination of biochemical and genomic studies, it was found that recurrent mutations in ARAF (e.g., Gly377 or Gly387 mutation) are one of the most significant factors that confer belvarafenib resistance. (Yen, I., et al, ARAF mutations confer resistance to the RAF inhibitor belvarafenib in melanoma, Nature, 2021 Vol 594 418-423).

Applicant surprisingly discovered that Compound I exhibit a 2— to 10-fold greater potency than other pan-RAF inhibitors (e.g., belvarafenib and naporafenib) in suppressing the kinase activity of ARAF, including ARAF mutants, whose expression is associated with the onset of resistance to belvarafenib. Without being bound by theory, these discoveries noted above provide for effective dosing regimens with Compound I in various forms of cancer harboring mutations in the MAPK pathway. In embodiments, the disclosed dosing regimens effectively treat various forms of cancer harboring mutations in the MAPK pathway while also minimizing side effects. In other words, the disclosed dosing regimens strike an acceptable benefit risk ratio.

In embodiments, the present application describes safe and effective dosing regimens of Compound I, or a pharmaceutically acceptable salt thereof, to provide antitumor activity in a subject with solid tumors harboring alterations in the MAPK pathway.

In one aspect, the present disclosure provides a method of treating cancer, comprising administering Compound I having a chemical structure:

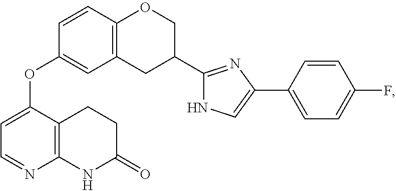

or an equivalent dose of a pharmaceutically acceptable salt thereof, to a subject in need thereof. In embodiments, Compound I is

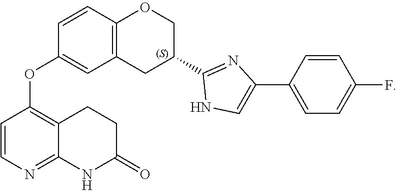

In embodiments, the Compound I of the present disclosure has an enantiomeric excess of at least 85%, at least 90%, at least 95%, or at least 99%.

In embodiments, the present disclosure provides a method of treating cancer harboring one or more ARAF mutants in a patient in need thereof, comprising administering about 5 mg to about 1500 mg (e.g., 5-500 mg or 30-500 mg) of Compound I or an equivalent dose of a pharmaceutically acceptable salt thereof, to the subject in need thereof. In embodiments, the present disclosure provides a method of treating cancer harboring one or more ARAF mutants in a patient in need thereof, comprising administering about 5 mg to about 500 mg of Compound I or an equivalent dose of a pharmaceutically acceptable salt thereof, to the subject in need thereof. In embodiments, the ARAF mutant comprises a mutation of G331C, A225V, F351L, P539H, Q347_A348del, N217I, G322S, A451T, R255Gfs(Ter)37, S214(Ter), P8Lfs(Ter)26, G387N/D/R/S, P462L, G377R, or a combination thereof. In embodiments, the ARAF mutant comprises a mutation of P462L, G377R, G387D, or a combination thereof. In embodiments, the Compound I or a pharmaceutically acceptable salt thereof is administered at a dose providing a steady state plasma concentration that is sufficient to inhibit the activity of the ARAF mutant.

In embodiments, the present disclosure provides a method of treating belvarafenib resistant cancer in a patient in need thereof, comprising administering about 5 mg to about 1500 mg of Compound I or an equivalent dose of a pharmaceutically acceptable salt thereof, to the subject in need thereof. In embodiments, the present disclosure provides a method of treating belvarafenib resistant cancer in a patient in need thereof, comprising administering about 5 mg to about 500 mg of Compound I or an equivalent dose of a pharmaceutically acceptable salt thereof, to the subject in need thereof. In embodiments, the belvarafenib resistant cancer expresses an ARAF mutant. In embodiments, the ARAF mutant comprises a mutation of P462L, G377R, G387D, or a combination thereof.

In embodiments, the present disclosure provides a method of treating naporafenib resistant cancer in a patient in need thereof, comprising administering about 5 mg to about 1500 mg of Compound I or an equivalent dose of a pharmaceutically acceptable salt thereof, to the subject in need thereof. In embodiments, the present disclosure provides a method of treating naporafenib resistant cancer in a patient in need thereof, comprising administering about 5 mg to about 500 mg of Compound I or an equivalent dose of a pharmaceutically acceptable salt thereof, to the subject in need thereof. In embodiments, the naporafenib resistant cancer expresses an ARAF mutant.

In embodiments, Compound I is:

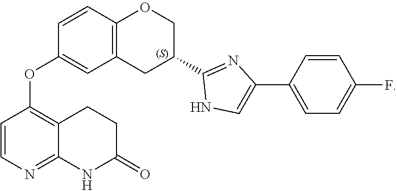

In embodiments, the cancer is advanced or metastatic cancer. In embodiments, the cancer comprises a solid tumor. In embodiments, the caner harbors alterations in the MAPK pathway. In embodiments, the harbors alterations in the MAPK pathway.

In embodiments, the therapeutically effective amount of Compound I, or an equivalent dose of a pharmaceutically acceptable salt thereof, administered to a subject ranges from 1 mg to about 2000 mg (e.g., about 1 mg to about 1900 mg, about 1 mg to about 1800 mg, about 1 mg to about 1700 mg, about 1 mg to about 1600 mg, about 1 mg to about 1500 mg, about 1 mg to about 1400 mg, about 1 mg to about 1300 mg, about 1 mg to about 1200 mg, about 1 mg to about 1100 mg, about 1 mg to about 1000 mg, about 1 mg to about 900 mg, about 1 mg to about 800 mg, about 1 mg to about 700 mg, about 1 mg to about 600 mg, about 1 mg to about 500 mg, about 1 mg to about 400 mg about 1 mg to about 300 mg, about 1 mg to about 200 mg, about 1 mg to about 100 mg, about 1 mg to about 90 mg, about 1 mg to about 80 mg, about 1 mg to about 70 mg, about 1 mg to about 60 mg, about 1 mg to about 50 mg, about 1 mg to about 40 mg, about 1 mg to about 30 mg, about 1 mg to about 20 mg, about 1 mg to about 10 mg, about 5 mg to about 2000 mg, about 5 mg to about 1900 mg, about 5 mg to about 1800 mg, about 5 mg to about 1700 mg, about 5 mg to about 1600 mg, about 5 mg to about 1500 mg, about 5 mg to about 1400 mg, about 5 mg to about 1300 mg, about 5 mg to about 1200 mg, about 5 mg to about 1100 mg, about 5 mg to about 1000 mg, about 5 mg to about 900 mg, about 5 mg to about 800 mg, about 5 mg to about 700 mg, about 5 mg to about 600 mg, about 5 mg to about 500 mg, about 5 mg to about 400 mg about 5 mg to about 300 mg, about 5 mg to about 200 mg, about 5 mg to about 100 mg, about 5 mg to about 90 mg, about 5 mg to about 80 mg, about 5 mg to about 70 mg, about 5 mg to about 60 mg, about 5 mg to about 50 mg, about 5 mg to about 40 mg, about 5 mg to about 30 mg, about 5 mg to about 20 mg, about 5 mg to about 10 mg, about 10 mg to about 2000 mg, about 10 mg to about 1900 mg, about 10 mg to about 1800 mg, about 10 mg to about 1700 mg, about 10 mg to about 1600 mg, about 10 mg to about 1500 mg, about 10 mg to about 1400 mg, about 10 mg to about 1300 mg, about 10 mg to about 1200 mg, about 10 mg to about 1100 mg, about 10 mg to about 1000 mg, about 10 mg to about 900 mg, about 10 mg to about 800 mg, about 10 mg to about 700 mg, about 10 mg to about 600 mg, about 10 mg to about 500 mg, about 10 mg to about 400 mg about 10 mg to about 300 mg, about 10 mg to about 200 mg, about 10 mg to about 100 mg, about 10 mg to about 90 mg, about 10 mg to about 80 mg, about 10 mg to about 70 mg, about 10 mg to about 60 mg, about 10 mg to about 50 mg, about 10 mg to about 40 mg, about 10 mg to about 30 mg, about 10 mg to about 20 mg, about 20 mg to about 2000 mg, about 20 mg to about 1900 mg, about 20 mg to about 1800 mg, about 20 mg to about 1700 mg, about 20 mg to about 1600 mg, about 20 mg to about 1500 mg, about 20 mg to about 1400 mg, about 20 mg to about 1300 mg, about 20 mg to about 1200 mg, about 20 mg to about 1100 mg, about 20 mg to about 1000 mg, about 20 mg to about 900 mg, about 20 mg to about 800 mg, about 20 mg to about 700 mg, about 20 mg to about 600 mg, about 20 mg to about 500 mg, about 20 mg to about 400 mg about 20 mg to about 300 mg, about 20 mg to about 200 mg, about 20 mg to about 100 mg, about 20 mg to about 90 mg, about 20 mg to about 80 mg, about 20 mg to about 70 mg, about 20 mg to about 60 mg, about 20 mg to about 50 mg, about 20 mg to about 40 mg, about 20 mg to about 30 mg, about 40 mg to about 2000 mg, about 40 mg to about 1900 mg, about 40 mg to about 1800 mg, about 40 mg to about 1700 mg, about 40 mg to about 1600 mg, about 40 mg to about 1500 mg, about 40 mg to about 1400 mg, about 40 mg to about 1300 mg, about 40 mg to about 1200 mg, about 40 mg to about 1100 mg, about 40 mg to about 1000 mg, about 40 mg to about 900 mg, about 40 mg to about 800 mg, about 40 mg to about 700 mg, about 40 mg to about 600 mg, about 40 mg to about 500 mg, about 40 mg to about 400 mg about 40 mg to about 300 mg, about 40 mg to about 200 mg, about 40 mg to about 100 mg, about 40 mg to about 90 mg, about 40 mg to about 80 mg, about 40 mg to about 70 mg, about 40 mg to about 60 mg, or about 40 mg to about 50 mg), including any values or ranges therebetween. In embodiments, about 5 mg to about 1500 mg of Compound I, or an equivalent dose of a pharmaceutically acceptable salt thereof, is administered. In embodiments, about 5 mg to about 500 mg of Compound I, or an equivalent dose of a pharmaceutically acceptable salt thereof, is administered. In embodiments, about 20 mg to about 1000 mg of Compound I, or an equivalent dose of a pharmaceutically acceptable salt thereof, is administered per day. In embodiments, about 40 mg to about 1000 mg of Compound I, or an equivalent dose of a pharmaceutically acceptable salt thereof, is administered per day. In embodiments, about 20 mg to about 500 mg of Compound I, or an equivalent dose of a pharmaceutically acceptable salt thereof, is administered per day. In embodiments, about 20 mg to about 300 mg of Compound I, or an equivalent dose of a pharmaceutically acceptable salt thereof, is administered per day. In embodiments, about 20 mg to about 200 mg of Compound I, or an equivalent dose of a pharmaceutically acceptable salt thereof, is administered per day.

In embodiments, the therapeutically effective amount of Compound I, or an equivalent dose of a pharmaceutically acceptable salt or stereoisomer, administered to the subject ranges from about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, about 1600 mg, about 1700 mg, about 1800 mg, about 1900, or about 2000 mg, including any values or ranges therebetween. In embodiments, about 10 mg, about 20 mg, about 30 mg, about 40 mg, about 50 mg, about 60 mg, about 75 mg, about 80 mg, about 100 mg, about 150 mg, about 200 mg, about 300 mg, about 500 mg, about 800 mg, about 1000 mg, about 1200 mg, or about 1500 mg of Compound I, or a pharmaceutically acceptable salt thereof, is administered. In embodiments, about 5 mg, about 10 mg, about 20 mg, about 25 mg, about 30 mg, about 40 mg, about 50 mg, about 60 mg, about 75 mg, about 80 mg, about 100 mg, about 150 mg, about 175 mg, about 200 mg, about 225 mg, about 275 mg, about 300 mg, about 325 mg, about 375 mg, about 425 mg, about 475 mg, about 500 mg, about 800 mg, about 1000 mg, about 1200 mg, or about 1500 mg of Compound I, or a pharmaceutically acceptable salt thereof, is administered. In embodiments, about 5 mg of Compound I, or an equivalent dose of a pharmaceutically acceptable salt thereof, is administered per day. In embodiments, about 20 mg of Compound I, or an equivalent dose of a pharmaceutically acceptable salt thereof, is administered per day. In embodiments, about 25 mg of Compound I, or an equivalent dose of a pharmaceutically acceptable salt thereof, is administered per day. In embodiments, about 30 mg of Compound I, or an equivalent dose of a pharmaceutically acceptable salt thereof, is administered per day. In embodiments, about 40 mg of Compound I, or an equivalent dose of a pharmaceutically acceptable salt thereof, is administered per day. In embodiments, about 50 mg of Compound I, or an equivalent dose of a pharmaceutically acceptable salt thereof, is administered per day. In embodiments, about 60 mg of Compound I, or an equivalent dose of a pharmaceutically acceptable salt thereof, is administered per day. In embodiments, about 75 mg of Compound I, or an equivalent dose of a pharmaceutically acceptable salt thereof, is administered per day. In embodiments, about 100 mg of Compound I, or an equivalent dose of a pharmaceutically acceptable salt thereof, is administered per day. In embodiments, about 150 mg of Compound I, or an equivalent dose of a pharmaceutically acceptable salt thereof, is administered per day. In embodiments, about 175 mg of Compound I, or an equivalent dose of a pharmaceutically acceptable salt thereof, is administered per day. In embodiments, about 200 mg of Compound I, or an equivalent dose of a pharmaceutically acceptable salt thereof, is administered per day. In embodiments, about 225 mg of Compound I, or an equivalent dose of a pharmaceutically acceptable salt thereof, is administered per day. In embodiments, about 275 mg of Compound I, or an equivalent dose of a pharmaceutically acceptable salt thereof, is administered per day. In embodiments, about 325 mg of Compound I, or an equivalent dose of a pharmaceutically acceptable salt thereof, is administered per day. In embodiments, about 375 mg of Compound I, or an equivalent dose of a pharmaceutically acceptable salt thereof, is administered per day. In embodiments, about 425 mg of Compound I, or an equivalent dose of a pharmaceutically acceptable salt thereof, is administered per day. In embodiments, about 475 mg of Compound I, or an equivalent dose of a pharmaceutically acceptable salt thereof, is administered per day. In embodiments, about 500 mg of Compound I, or an equivalent dose of a pharmaceutically acceptable salt thereof, is administered per day. In embodiments, about 1000 mg of Compound I, or an equivalent dose of a pharmaceutically acceptable salt thereof, is administered per day.

In embodiments, Compound I, or an equivalent dose of a pharmaceutically acceptable salt thereof, is administered once a day or twice a day. In embodiments, about 5 mg of Compound I, or an equivalent dose of a pharmaceutically acceptable salt thereof, is administered once a day (i.e., a total daily dose of 5 mg). In embodiments, about 20 mg of Compound I, or an equivalent dose of a pharmaceutically acceptable salt thereof, is administered once a day (i.e., a total daily dose of 20 mg). In embodiments, about 25 mg of Compound I, or an equivalent dose of a pharmaceutically acceptable salt thereof, is administered once a day (i.e., a total daily dose of 25 mg). In embodiments, about 30 mg of Compound I, or an equivalent dose of a pharmaceutically acceptable salt thereof, is administered once a day (i.e., a total daily dose of 30 mg). In embodiments, about 40 mg of Compound I, or an equivalent dose of a pharmaceutically acceptable salt thereof, is administered once a day (i.e., a total daily dose of 40 mg). In embodiments, about 50 mg of Compound I, or an equivalent dose of a pharmaceutically acceptable salt thereof, is administered once a day (i.e., a total daily dose of 50 mg). In embodiments, about 75 mg of Compound I, or an equivalent dose of a pharmaceutically acceptable salt thereof, is administered once a day (i.e., a total daily dose of 75 mg). In embodiments, about 100 mg of Compound I, or an equivalent dose of a pharmaceutically acceptable salt thereof, is administered once a day (i.e., a total daily dose of 100 mg). In embodiments, about 150 mg of Compound I, or an equivalent dose of a pharmaceutically acceptable salt thereof, is administered once a day (i.e., a total daily dose of 150 mg). In embodiments, about 175 mg of Compound I, or an equivalent dose of a pharmaceutically acceptable salt thereof, is administered once a day (i.e., a total daily dose of 175 mg). In embodiments, about 200 mg of Compound I, or an equivalent dose of a pharmaceutically acceptable salt thereof, is administered once a day (i.e., a total daily dose of 200 mg). In embodiments, about 225 mg of Compound I, or an equivalent dose of a pharmaceutically acceptable salt thereof, is administered once a day (i.e., a total daily dose of 225 mg). In embodiments, about 275 mg of Compound I, or an equivalent dose of a pharmaceutically acceptable salt thereof, is administered once a day (i.e., a total daily dose of 275 mg). In embodiments, about 325 mg of Compound I, or an equivalent dose of a pharmaceutically acceptable salt thereof, is administered once a day (i.e., a total daily dose of 325 mg). In embodiments, about 375 mg of Compound I, or an equivalent dose of a pharmaceutically acceptable salt thereof, is administered once a day (i.e., a total daily dose of 375 mg). In embodiments, about 425 mg of Compound I, or an equivalent dose of a pharmaceutically acceptable salt thereof, is administered once a day (i.e., a total daily dose of 425 mg). In embodiments, about 475 mg of Compound I, or an equivalent dose of a pharmaceutically acceptable salt thereof, is administered once a day (i.e., a total daily dose of 475 mg). In embodiments, about 500 mg of Compound I, or an equivalent dose of a pharmaceutically acceptable salt thereof, is administered once a day (i.e., a total daily dose of 500 mg).

In embodiments, about 5 mg of Compound I, or an equivalent dose of a pharmaceutically acceptable salt thereof, is administered twice a day (i.e., a total daily dose of 10 mg). In embodiments, about 20 mg of Compound I, or an equivalent dose of a pharmaceutically acceptable salt thereof, is administered twice a day (i.e., a total daily dose of 40 mg). In embodiments, about 25 mg of Compound I, or an equivalent dose of a pharmaceutically acceptable salt thereof, is administered twice a day (i.e., a total daily dose of 50 mg). In embodiments, about 30 mg of Compound I, or an equivalent dose of a pharmaceutically acceptable salt thereof, is administered twice a day (i.e., a total daily dose of 60 mg). In embodiments, about 40 mg of Compound I, or an equivalent dose of a pharmaceutically acceptable salt thereof, is administered twice a day (i.e., a total daily dose of 80 mg). In embodiments, about 50 mg of Compound I, or an equivalent dose of a pharmaceutically acceptable salt thereof, is administered twice a day (i.e., a total daily dose of 100 mg). In embodiments, about 75 mg of Compound I, or an equivalent dose of a pharmaceutically acceptable salt thereof, is administered twice a day (i.e., a total daily dose of 150 mg). In embodiments, about 100 mg of Compound I, or an equivalent dose of a pharmaceutically acceptable salt thereof, is administered twice a day (i.e., a total daily dose of 200 mg). In embodiments, about 150 mg of Compound I, or an equivalent dose of a pharmaceutically acceptable salt thereof, is administered twice a day (i.e., a total daily dose of 300 mg). In embodiments, about 175 mg of Compound I, or an equivalent dose of a pharmaceutically acceptable salt thereof, is administered twice a day (i.e., a total daily dose of 350 mg). In embodiments, about 200 mg of Compound I, or an equivalent dose of a pharmaceutically acceptable salt thereof, is administered twice a day (i.e., a total daily dose of 400 mg). In embodiments, about 225 mg of Compound I, or an equivalent dose of a pharmaceutically acceptable salt thereof, is administered twice a day (i.e., a total daily dose of 450 mg). In embodiments, about 250 mg of Compound I, or an equivalent dose of a pharmaceutically acceptable salt thereof, is administered twice a day (i.e., a total daily dose of 500 mg). In embodiments, about 275 mg of Compound I, or an equivalent dose of a pharmaceutically acceptable salt thereof, is administered twice a day (i.e., a total daily dose of 550 mg). In embodiments, about 325 mg of Compound I, or an equivalent dose of a pharmaceutically acceptable salt thereof, is administered twice a day (i.e., a total daily dose of 650 mg). In embodiments, about 350 mg of Compound I, or an equivalent dose of a pharmaceutically acceptable salt thereof, is administered twice a day (i.e., a total daily dose of 700 mg). In embodiments, about 375 mg of Compound I, or an equivalent dose of a pharmaceutically acceptable salt thereof, is administered twice a day (i.e., a total daily dose of 750 mg). In embodiments, about 425 mg of Compound I, or an equivalent dose of a pharmaceutically acceptable salt thereof, is administered twice a day (i.e., a total daily dose of 850 mg). In embodiments, about 475 mg of Compound I, or an equivalent dose of a pharmaceutically acceptable salt thereof, is administered twice a day (i.e., a total daily dose of 950 mg). In embodiments, about 500 mg of Compound I, or an equivalent dose of a pharmaceutically acceptable salt thereof, is administered twice a day (i.e., a total daily dose of 1000 mg). In embodiments, Compound I, or an equivalent dose of a pharmaceutically acceptable salt thereof, is administered with any one of the following dosing regimens.

Compound I dosing regimens: each of following
dose administered once daily or twice daily

| | | | | | | | | | |
|---|---|---|---|---|---|---|---|---|---|
| 20 | mg | 125 | mg | 230 | mg | 350 | mg | 460 | mg |
| 25 | mg | 130 | mg | 240 | mg | 360 | mg | 470 | mg |
| 30 | mg | 140 | mg | 250 | mg | 370 | mg | 475 | mg |
| 40 | mg | 150 | mg | 260 | mg | 375 | mg | 480 | mg |
| 50 | mg | 160 | mg | 270 | mg | 380 | mg | 490 | mg |
| 60 | mg | 170 | mg | 280 | mg | 390 | mg | 500 | mg |
| 70 | mg | 175 | mg | 290 | mg | 400 | mg | 550 | mg |
| 75 | mg | 180 | mg | 300 | mg | 410 | mg | 600 | mg |
| 80 | mg | 190 | mg | 310 | mg | 420 | mg | 700 | mg |
| 90 | mg | 200 | mg | 320 | mg | 425 | mg | 750 | mg |
| 100 | mg | 210 | mg | 325 | mg | 430 | mg | 800 | mg |
| 110 | mg | 220 | mg | 330 | mg | 440 | mg | 900 | mg |
| 120 | mg | 225 | mg | 340 | mg | 450 | mg | 1000 | mg |

In embodiments, the cancer is Barret's adenocarcinoma; biliary tract carcinomas; breast cancer; cervical cancer; cholangiocarcinoma; central nervous system tumors; primary CNS tumors; glioblastomas; astrocytomas; glioblastoma multiforme; ependymomas; secondary CNS tumors (metastases to the central nervous system of tumors originating outside of the central nervous system); brain tumors; brain metastases; colorectal cancer; large intestinal colon carcinoma; gastric cancer; carcinoma of the head and neck; squamous cell carcinoma of the head and neck; acute lymphoblastic leukemia; acute myelogenous leukemia (AML); myelodysplastic syndromes; chronic myelogenous leukemia; Hodgkin's lymphoma; non-Hodgkin's lymphoma; megakaryoblastic leukemia; multiple myeloma; erythroleukemia; hepatocellular carcinoma; lung cancer; small cell lung cancer; non-small cell lung cancer; ovarian cancer; endometrial cancer; pancreatic cancer; pituitary adenoma; prostate cancer; renal cancer; melanoma; neurofibromatosis type 1 (NF1); neurofibromatosis type 2 (NF2); esophageal cancer; bladder cancer; mesothelioma; or thyroid cancer.

In embodiments, the cancer is non-small cell lung cancer (NSCLC). In embodiments, the cancer is colorectal cancer. In embodiments, the cancer is melanoma. In embodiments, the cancer is pancreatic cancer. In embodiments, the cancer is advanced or metastatic cancer. In some embodiments, melanoma is metastatic melanoma. In embodiments, the cancer is thyroid cancer. In embodiments, the thyroid cancer is papillary, follicular, Hürthle cell, medullary, or anaplastic thyroid cancer.

In embodiments, the cancer comprises a solid tumor harboring one or more alterations in the mitogen activated protein kinase (MAPK) pathway. In embodiments, the one or more alterations comprises a KRAS mutation, a NRAS mutation, a HRAS mutation, a BRAF mutation, a BRAF fusion, a BRAF translocation, an ARAF mutation, RAF mutation, an increased receptor tyrosine kinase (RTK) signaling, RAS gene amplification, RAF gene amplification, or a combination thereof.

In embodiments, the KRAS mutation comprises G12C, G12D, G12R, G12V, G12W, G13C, G13D, G13R, G13V, G13W, Q61R, Q61K, Q61L, Q61H, Q61P, A146P, A146T, A146V, A59G, A59T, R68S, H95D, H95Q, H95R, Y96C, or a combination thereof. In embodiments, the NRAS mutation comprises Q61R, Q61K, Q61L, Q61H, Q61P, G12S, G12D, G12C, G12V, G12A, G12R, G13V, G13R, G13D, G13C, or a combination thereof. In embodiments, the HRAS mutation comprises Q61R, Q61K, Q61L, G12S, G12D, G12C, G12V, G12A, G12R, G13V, G13R, G13D, G13C, or a combination thereof.

In embodiments, the ARAF mutation comprises G331C, A225V, F351L, P539H, Q347_A348del, N217I, G322S, A451T, R255Gfs(Ter)37, S214(Ter), P8Lfs(Ter)26, G387N/D/R/S, P462L, G377R, or a combination thereof.

In embodiments, the BRAF mutation comprises Class 1 BRAF mutation, Class 2 BRAF mutation, Class 3 BRAF mutation, or a combination thereof. In embodiments, the BRAF mutation comprises Class 2 or 3 BRAF mutation. In embodiments, the BRAF mutation comprises V600E, V600M, V600G, V600K, V600R, V600D, G469A, G469V, G469R, K601E, K601N, K601T, L597Q, L597V, G464V, G464E, D594N, D594G, D594A, D594H, G466A, G466V, G466E, N581S, N581I, G469E, S467L, G596D, G596R, F595L, V459L, D287H or a combination thereof.

In embodiments, the RAF mutation comprises S257L/W, S259F, R391S/W, or a combination thereof. In embodiments, the one or more alterations comprise NRAS Q61K, KRAS G12C, KRAS G13D, KRAS Q61K, or a combination thereof.

In embodiments, Compound I, or a pharmaceutically acceptable salt thereof, is administered for at least about 14 days, e.g., 2 weeks, or 3 weeks, or 4 weeks, or 5 weeks, or 6 weeks, or 7 weeks, or 8 weeks, or 9 weeks, or 10 weeks, or 11 weeks, or 12 weeks, or 13 weeks, or 14 weeks, or 15 weeks, or 16 weeks, or 17 weeks, or 18 weeks, or 19 weeks, or 20 weeks, or 21 weeks, or 22 weeks, or 23 weeks, or 24 weeks, or 25 weeks, or 26 weeks, or 27 weeks, or 28 weeks, or 29 weeks, or 30 weeks, or 31 weeks, or 32 weeks, or 33 weeks, or 34 weeks, or 35 weeks, or 36 weeks, or 37 weeks, or 38 weeks, or 39 weeks, or 40 weeks, or 41 weeks, or 42 weeks, or 43 weeks, or 44 weeks, or 45 weeks, or 46 weeks, or 47 weeks, or 48 weeks, or 49 weeks, or 50 weeks, or 51 weeks, or 52 weeks, or 53 weeks, or 54 weeks, or 55 weeks, or 56 weeks, or 57 weeks, or 58 weeks, or 59 weeks, or 60 weeks, or 61 weeks, or 62 weeks, or 63 weeks, or 64 weeks, or 65 weeks, or 66 weeks, or 67 weeks, or 68 weeks, or 69 weeks, or 70 weeks, or 71 weeks, or 72 weeks, or 73 weeks, or 74 weeks, or 75 weeks, or 76 weeks, or 77 weeks, or 78 weeks, or 79 weeks, or 80 weeks, or 81 weeks, or 82 weeks, or 83 weeks, or 84 weeks, or 85 weeks, or 86 weeks, or 87 weeks, or 88 weeks, or 89 weeks, or 90 weeks, or 91 weeks, or 92 weeks, or 93 weeks, or 94 weeks, or 95 weeks, or 96 weeks, or 97 weeks, or 98 weeks, or 99 weeks, or 100 weeks, or 101 weeks, or 102 weeks, or 103 weeks, or 104 weeks, 3 years, 4 years, 5 years, 6 years, 7 years, 8 years, 9 years, 10 years, 11 years, 12, years, 13 years, 14 years, 15 years, 16 years, 17 years, 18 years, 19 years, 20 years, or more.

In embodiments, the present disclosure provides a method of treating cancer harboring one or more alterations in the mitogen activated protein kinase (MAPK) pathway in a subject, comprising: detecting the one or more alterations in the MAPK pathway; and then administering about 5 mg to about 1500 mg of Compound I having a chemical structure:

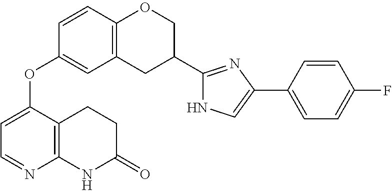

or an equivalent dose of a pharmaceutically acceptable salt thereof, to the subject. In embodiments, the present disclosure provides a method of treating cancer harboring one or more alterations in the mitogen activated protein kinase (MAPK) pathway in a subject, comprising: detecting the one or more alterations in the MAPK pathway; and then administering about 5 mg to about 500 mg of Compound I or an equivalent dose of a pharmaceutically acceptable salt thereof, to the subject.

In embodiments, Compound I is

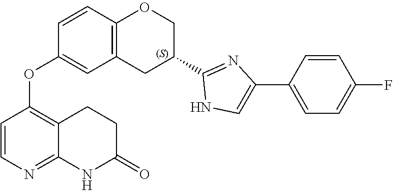

In embodiments, the Compound I of the present disclosure has an enantiomeric excess of at least 85%, at least 90%, at least 95%, or at least 99%.

In embodiments, the one or more alterations comprises a KRAS mutation, a NRAS mutation, a HRAS mutation, a BRAF mutation, a BRAF fusion, a BRAF translocation, an ARAF mutation, RAF mutation, an increased receptor tyrosine kinase (RTK) signaling, RAS gene amplification, RAF gene amplification, or a combination thereof.

In embodiments, the KRAS mutation comprises G12C, G12D, G12R, G12V, G12W, G13C, G13D, G13R, G13V, G13W, Q61R, Q61K, Q61L, Q61H, Q61P, A146P, A146T, A146V, A59G, A59T, R68S, H95D, H95Q, H95R, Y96C, or a combination thereof. In embodiments, the NRAS mutation comprises Q61R, Q61K, Q61L, Q61H, Q61P, G12S, G12D, G12C, G12V, G12A, G12R, G13V, G13R, G13D, G13C, or a combination thereof. In embodiments, the HRAS mutation comprises Q61R, Q61K, Q61L, G12S, G12D, G12C, G12V, G12A, G12R, G13V, G13R, G13D, G13C, or a combination thereof.

In embodiments, the ARAF mutation comprises G331C, A225V, F351L, P539H, Q347_A348del, N217I, G322S, A451T, R255Gfs(Ter)37, S214(Ter), P8Lfs(Ter)26, G387N/D/R/S, P462L, G377R, or a combination thereof.

In embodiments, the BRAF mutation comprises Class 1 BRAF mutation, Class 2 BRAF mutation, Class 3 BRAF mutation, or a combination thereof. In embodiments, the BRAF mutation comprises Class 2 or 3 BRAF mutation. In embodiments, the BRAF mutation comprises V600E, V600M, V600G, V600K, V600R, V600D, G469A, G469V, G469R, K601E, K601N, K601T, L597Q, L597V, G464V, G464E, D594N, D594G, D594A, D594H, G466A, G466V, G466E, N581S, N581I, G469E, S467L, G596D, G596R, F595L, V459L, D287H or a combination thereof.

In embodiments, the RAF mutation comprises S257L/W, S259F, R391S/W, or a combination thereof. In embodiments, the one or more alterations comprise NRAS Q61K, KRAS G12C, KRAS G13D, KRAS Q61K, or a combination thereof.

In embodiments, the one or more alterations are detected and quantified using suitable method, for instance, by using real-time quantitative polymerase chain reaction (RT-PCR), immunohistochemistry, and/or Western blot, as described in U.S. Pat. Nos. 9,567,640 and 8,232,062, herein incorporated by reference in their entireties.

### Pharmaceutical Compositions and Formulations

In embodiments, Compound I, or a pharmaceutically acceptable salt thereof, is present in a pharmaceutical composition. In embodiments, the pharmaceutical composition comprises a therapeutically effective amount of Compound I, or an equivalent dose of pharmaceutically acceptable salt thereof, and a pharmaceutically acceptable carrier or excipient. In embodiments, the pharmaceutical compositions of the present disclosure are formulated for oral, intravenous, intramuscular, subcutaneous or parenteral administration.

In embodiments, Compound I or the pharmaceutical compositions of the present disclosure are administered by oral administration in the form of tablets, capsules, syrups, powders or granules. In embodiments, Compound I, or an equivalent dose of a pharmaceutically acceptable salt thereof, is orally administered in a pharmaceutical formulation. In embodiments, the pharmaceutical composition may be a solid, powder, liquid and a gel. In embodiments, the pharmaceutical is a solid (e.g., a powder, tablet, a capsule, granulates, and/or aggregates). In certain of such embodiments, the solid pharmaceutical composition comprises one or more excipients known in the art, including, but not limited to, starches, sugars, diluents, granulating agents, lubricants, binders, and disintegrating agents. In embodiments, the pharmaceutical formulation is a tablet or a capsule. In embodiments, the pharmaceutical formulation is a capsule.

For oral administration, Compound I, or pharmaceutically acceptable salt or stereoisomer thereof, may be admixed with an adjuvant or a carrier, for example, lactose, saccharose, sorbitol, mannitol; a starch, for example, potato starch, corn starch or amylopectin; a cellulose derivative; a binder, for example, gelatin or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatin capsules, Compound I or salt thereof may be admixed with, for example, a vegetable oil or polyethylene glycol. Hard gelatin capsules may contain granules of the compound using either of the above-mentioned excipients for tablets. Also, liquid or semisolid formulations of Compound I or salt thereof may be filled into hard gelatin capsules. Liquid preparations for oral application may be in the form of syrups or suspensions, for example, solutions containing Compound I or salt thereof, the balance being sugar and a mixture of ethanol, water, glycerol and propylene glycol. Such liquid preparations may optionally contain coloring agents, flavoring agents, sweetening agents (such as saccharine), preservative agents and/or carboxymethylcellulose as a thickening agent or other excipients known to those skilled in art.

In embodiments, the pharmaceutical formulation comprises about 1 mg to about 500 mg of Compound I, or an equivalent dose of a pharmaceutically acceptable salt thereof, for example, about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, or about 500 mg, including any values or ranges therebetween. In embodiments, the pharmaceutical formulation comprises about 5 mg to about 200 mg of Compound I, or an equivalent dose of a pharmaceutically acceptable salt thereof. In embodiments, the pharmaceutical formulation comprises about 5 mg to about 100 mg of Compound I, or an equivalent dose of a pharmaceutically acceptable salt thereof. In embodiments, the pharmaceutical formulation comprises about 5 mg to about 50 mg of Compound I, or an equivalent dose of a pharmaceutically acceptable salt thereof. In embodiments, the pharmaceutical formulation comprises about 10 mg to about 25 mg of Compound I, or an equivalent dose of a pharmaceutically acceptable salt thereof. In embodiments, the pharmaceutical formulation comprises about 25 mg of Compound I, or an equivalent dose of a pharmaceutically acceptable salt thereof.

In embodiments, the pharmaceutical composition may contain additional materials useful in physically formulating various dosage forms of the compositions of the present disclosure, such as dyes, flavoring agents, preservatives, antioxidants, opacifiers, thickening agents, stabilizers lubricants, wetting agents, emulsifiers, salts for influencing osmotic pressure, buffers, colorings, flavorings and/or aromatic substances, but not additional anti-tumor agent.

### Combination Therapies

In embodiments, Compound I, or a pharmaceutically acceptable salt thereof, is administered in combination with one or more therapeutic agents.

In embodiments, Compound I, or a pharmaceutically acceptable salt thereof, is administered in combination with one or more targeted therapy agents. In embodiments, Compound I, or a pharmaceutically acceptable salt thereof, is administered in combination with immune effector cells (e.g., T cells, NK cells) that are engineered to contain one or more chimeric antigen receptors (CARs) and one or more targeted therapy agents. A plurality of types of cells may be contemplated as a targeted therapy agent, for example, CAR T cells, genetically modified T cells, tumor infiltrating lymphocytes (TIL), vaccines, and/or natural killer (NK) cells. In embodiments, the one or more targeted therapy agents comprise a gene therapy agent, including for example an agent used in approaches to replace aberrant genes such as aberrant p53 or aberrant BRCA1 or BRCA2.

In embodiments, the one or more targeted therapy agents comprise a ALK inhibitor (for example alectinib, brigatinib, ceritinib, and crizotinib); antiogenesis inhibitor (for example axitinib, cabozantinib, cabozantinib, levatinib, pazopanib, regorafenib, sorafenib, sunitinib, vandetanib, and ziv-aflibercept); a BCR-ABL kinase inhibitor (for example bosutinib, dasatinib, imatinib mesylate, nilotinib, and ponatinib); a BRAF and MEK inhibitor (for example binimetinib, cobimetinib, dabrafenib, trametinib, and vemurafenib); a BTK inhibitor (for example acalabrutinib and ibrutinib); cyclin-dependent kinase inhibitor (for example abemaciclib, palbociclib, and ribociclib); a EGFR inhibitor (for example afatinib, erlotinib, gefitinib, lapatinib, neratinib, and osimertinib); a PARP inhibitor (for example olaparib, niraparib, and rucaparib); and a PI3K inhibitor (for example duvelisib, copanlisib, perifosine, and idelalisib), or a combination thereof.

In embodiments, the one or more therapeutic agents comprise an anti-tumor agent. In embodiments, the additional therapeutic agent is an antiproliferative/antineoplastic drug. In embodiments, antiproliferative/antineoplastic drug is an alkylating agent (for example cis-platin, oxaliplatin, carboplatin, cyclophosphamide, nitrogen mustard, bendamustin, melphalan, chlorambucil, busulphan, temozolamide and nitrosoureas); an antimetabolite (for example gemcitabine and antifolates such as fluoropyrimidines like 5-fluorouracil and tegafur, raltitrexed, methotrexate, pemetrexed, cytosine arabinoside, and hydroxyurea); an antibiotic (for example anthracyclines like adriamycin, bleomycin, doxorubicin, daunomycin, epirubicin, idarubicin, mitomycin-C, dactinomycin and mithramycin); an antimitotic agent (for example vinca alkaloids like vincristine, vinblastine, vindesine and vinorelbine and taxoids like taxol and taxotere and polokinase inhibitors); a proteasome inhibitor, for example carfilzomib and bortezomib; interferon therapy; or topoisomerase inhibitor (for example epipodophyllotoxins like etoposide and teniposide, amsacrine, topotecan, mitoxantrone and camptothecin).

In embodiments, the one or more therapeutic agents comprise a cytostatic agent. In embodiments, cytostatic agent is an antiestrogen (for example tamoxifen, fulvestrant, toremifene, raloxifene, droloxifene and iodoxyfene), an antiandrogen (for example bicalutamide, flutamide, nilutamide and cyproterone acetate), an LHRH antagonist or LHRH agonist (for example goserelin, leuprorelin and buserelin), a progestogen (for example megestrol acetate), an aromatase inhibitor (for example as anastrozole, letrozole, vorazole and exemestane) or an inhibitor of 5a-reductase such as finasteride.

In embodiments, the one or more therapeutic agents comprise an anti-invasion agent. In embodiments, the anti-invasion agent is dasatinib and bosutinib (SKI-606), metalloproteinase inhibitor, or inhibitor of urokinase plasminogen activator receptor function or antibody to Heparanase.

In embodiments, the one or more therapeutic agents comprise an inhibitor of growth factor function. In embodiments, the inhibitor of growth factor function is growth factor antibody and growth factor receptor antibody, for example the anti-erbB2 antibody trastuzumab [Herceptin™], the anti-EGFR antibody panitumumab, the anti-erbB1 antibody cetuximab, tyrosine kinase inhibitor, for example inhibitors of the epidermal growth factor family (for example EGFR family tyrosine kinase inhibitor such as gefitinib, erlotinib and 6-acrylamido-N-(3-chloro-4-fluorophenyl)-7-(3-morpholinopropoxy)-quinazolin-4-amine (CI 1033), erbB2 tyrosine kinase inhibitor such as lapatinib); inhibitor of the hepatocyte growth factor family; inhibitor of the insulin growth factor family; modulator of protein regulators of cell apoptosis (for example Bcl-2 inhibitors); inhibitor of the platelet-derived growth factor family such as imatinib and/or nilotinib (AMN107); inhibitor of serine/threonine kinases (for example Ras/RAF signaling inhibitors such as farnesyl transferase inhibitor, for example sorafenib, tipifarnib and lonafarnib), inhibitor of cell signaling through MEK and/or AKT kinase, c-kit inhibitor, abl kinase inhibitor, Plt3 kinase inhibitor, CSF-1R kinase inhibitor, IGF receptor, kinase inhibitor; aurora kinase inhibitor or cyclin dependent kinase inhibitor such as CDK2 and/or CDK4 inhibitor.

In embodiments, the one or more therapeutic agents comprise an antiangiogenic agent. In embodiments, the antiangiogenic agent inhibits the effects of vascular endothelial growth factor, for example the anti-vascular endothelial cell growth factor antibody bevacizumab (Avastin™); thalidomide; lenalidomide; and for example, a VEGF receptor tyrosine kinase inhibitor such as vandetanib, vatalanib, sunitinib, axitinib and pazopanib.

In embodiments, the one or more therapeutic agents comprise a cytotoxic agent. In embodiments, the cytotoxic agent is fludarabine (fludara), cladribine, or pentostatin (Nipent™).

In embodiments, the one or more therapeutic agents comprise a steroid. In embodiments, the steroid is corticosteroid, including glucocorticoid and mineralocorticoid, for example aclometasone, aclometasone dipropionate, aldosterone, amcinonide, beclomethasone, beclomethasone dipropionate, betamethasone, betamethasone dipropionate, betamethasone sodium phosphate, betamethasone valerate, budesonide, clobetasone, clobetasone butyrate, clobetasol propionate, cloprednol, cortisone, cortisone acetate, cortivazol, deoxycortone, desonide, desoximetasone, dexamethasone, dexamethasone sodium phosphate, dexamethasone isonicotinate, difluorocortolone, fluclorolone, flumethasone, flunisolide, fluocinolone, fluocinolone acetonide, fluocinonide, fluocortin butyl, fluorocortisone, fluorocortolone, fluocortolone caproate, fluocortolone pivalate, fluorometholone, fluprednidene, fluprednidene acetate, flurandrenolone, fluticasone, fluticasone propionate, halcinonide, hydrocortisone, hydrocortisone acetate, hydrocortisone butyrate, hydrocortisone aceponate, hydrocortisone buteprate, hydrocortisone valerate, icomethasone, icomethasone enbutate, meprednisone, methylprednisolone, mometasone paramethasone, mometasone furoate monohydrate, prednicarbate, prednisolone, prednisone, tixocortol, tixocortol pivalate, triamcinolone, triamcinolone acetonide, triamcinolone alcohol and their respective pharmaceutically acceptable derivatives. A combination of steroids may be used, for example a combination of two or more steroids as described herein.

In embodiments, the one or more therapeutic agents comprise an immunotherapeutic agent. In embodiments, the immunotherapeutic agent is interferon or colony stimulating factor. In embodiments, the immunotherapeutic agent is a PD-1 or PDL-1 inhibitor, (for example nivolumab, cemilimab, dostarimab, sintilimab, atezolizumab, avelumab, and durvalumab); a CTLA-4 inhibitor (for example ipilimumab and tremelimumab); or a LAG-3 inhibitor such as relatilimab.

In embodiments, the one or more therapeutic agents comprise an antibody-drug conjugate (for example trastuzumab emtansine, trastuzumab deruxtecan, enfortumab vedotin, and sacituzumab govitecan).

In embodiments, Compound I, or a pharmaceutically acceptable salt thereof, is simultaneously co-administered with one or more therapeutic agents. In embodiments, Compound I, or a pharmaceutically acceptable salt thereof, is sequentially administered with one or more therapeutic agents.

### Clinical Outcomes

In embodiments, the method of the present disclosure reduces size of one or more tumors (referred to as “tumor size” or “tumor volume”) compared to the tumor size prior to administration. In embodiments, the treatment reduces tumor size by about 10% to about 95%, about 15% to about 95%, about 20% to about 95%, about 30% to about 95%, about 40% to about 95%, about 50% to about 95%, or at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 95%, or at least about 100%, including any values or ranges therebetween, compared to the tumor size prior to administration. In embodiments, the changes in tumor size are detected and quantified by response evaluation criteria in solid tumors (RECIST), using a Computed Tomography (CT) scan, a Positron Emission Tomography-Computed Tomography (PET/CT) scan, an MRI scan, or an X-ray scan.

In embodiments, the method of the present disclosure provides treatment that stabilizes the tumor size. In embodiments, the treatment maintains the size of the tumor such that the tumor after administration has the same as the size of the tumor prior to administration. As used herein stabilizes refers to a tumor that changes in size by +5% after administration compared to tumor size prior to administration.

In embodiments, the treatment increases overall response rate (ORR) compared to the ORR of an otherwise similar patient with cancer that is not treated with Compound I, or a pharmaceutically acceptable salt thereof. In embodiments, the treatment increases ORR by at least about 1%, at least about 2%, at least about 3%, at least about 4%, at least about 5%, at least about 6%, at least about 7%, at least about 8%, at least about 9%, or at least about 10%, including any values or ranges therebetween. In embodiments, treating according to the methods of the disclosure results in an increase in the ORR of about 10%, about 20%, about 30%, about 40%, about 50%, about 60%, about 70%, about 80%, about 90%, about 100%, about 150%, about 200%, about 250%, about 300%, about 350%, about 400%, about 450%, about 500%, about 550%, about 600%, about 650%, about 700%, about 750%, about 800%, about 850%, about 900%, about 950%, or about 1000% or more, including all values and ranges in between, compared to the ORR of an otherwise similar patient with cancer that is not treated with Compound I, or a pharmaceutically acceptable salt thereof. In embodiments, the treatment increases ORR by about 10%, about 20%, about 30%, about 40%, about 50%, about 60%, about 70%, about 80%, about 90%, or about 100%, including any values or ranges therebetween, compared to the ORR of an otherwise similar patient with cancer that is not treated with Compound I, or a pharmaceutically acceptable salt thereof. In embodiments, the treatment increases ORR by at least about 5%, compared to the ORR of an otherwise similar patient with cancer that is not treated with Compound I, or a pharmaceutically acceptable salt thereof. In embodiments, the treatment increases ORR by about 5% to about 1000%, about 10% to about 1000%, about 20% to about 1000%, about 30% to about 1000%, about 40% to about 1000%, about 50% to about 1000%, about 60% to about 1000%, about 70% to about 1000%, about 80% to about 1000%, about 90% to about 1000%, about 100% to about 1000%, about 150% to about 1000%, about 200% to about 1000%, about 250% to about 1000%, about 300% to about 1000%, about 350% to about 1000%, about 400% to about 1000%, about 450% to about 1000%, about 500% to about 1000%, about 600% to about 1000%, about 700% to about 1000%, about 800% to about 1000%, about 900% to about 1000%, including all values and ranges in between, compared to the ORR of an otherwise similar patient with cancer that is not treated with Compound I, or a pharmaceutically acceptable salt thereof.

In embodiments, the treatment increases progression free survival (PFS), compared to the PFS of an otherwise similar patient with cancer that is not treated with Compound I, or a pharmaceutically acceptable salt thereof. In embodiments, the treatment increases PFS by at least about 1 month, at least about 2 months, at least about 3 month, at least about 4 months, at least about 5 month, at least about 6 months, at least about 7 month, at least about 8 months, at least about 9 month, at least about 10 months, at least about 11 month, at least about 12 months, at least about 13 month, at least about 14 months, at least about 15 month, at least about 16 months, at least about 17 month, at least about 18 months, at least about 19 month, at least about 20 months, at least about 21 month, at least about 22 months, at least about 23 month, or at least about 24 months, about 3 years, about 4 years, about 5 years, about 6 years, about 7 years, about 8 years, about 9 years, or about 10 years, including any values and ranges therebetween, compared to the PFS of an otherwise similar patient with cancer that is not treated with Compound I, or a pharmaceutically acceptable salt thereof. In embodiments, the treatment increases PFS by at least about 3 months, compared to the PFS of an otherwise similar patient with cancer that is not treated with Compound I, or a pharmaceutically acceptable salt thereof. In embodiments, the treatment increases PFS by at least about 6 months, at least 12 months, at least about 18 months, or at least about 24 months, compared to the PFS of an otherwise similar patient with cancer that is not treated with Compound I, or a pharmaceutically acceptable salt thereof. In embodiments, the treatment increases PFS by about 1 month to about 5 years, about 2 month to about 5 years, about 4 month to about 5 years, about 6 month to about 5 years, about 8 month to about 5 years, about 10 month to about 5 years, about 12 month to about 5 years, about 18 month to about 5 years, about 24 month to about 5 years, about 3 years to about 5 years, about 4 years to about 5 years, including any values and ranges therebetween, compared to the PFS of an otherwise similar patient with cancer that is not treated with Compound I, or a pharmaceutically acceptable salt thereof.

In embodiments, treating according to the methods of the disclosure increases PFS by about 10%, about 20%, about 30%, about 40%, about 50%, about 60%, about 70%, about 80%, about 90%, about 100%, about 150%, about 200%, about 250%, about 300%, about 350%, about 400%, about 450%, about 500%, about 550%, about 600%, about 650%, about 700%, about 750%, about 800%, about 850%, about 900%, about 950%, or about 1000% or more, including all values and ranges in between, compared to the PFS of an otherwise similar patient with cancer that is not treated with Compound I. In embodiments, the treatment increases PFS by about 5% to about 1000%, about 10% to about 1000%, about 20% to about 1000%, about 30% to about 1000%, about 40% to about 1000%, about 50% to about 1000%, about 60% to about 1000%, about 70% to about 1000%, about 80% to about 1000%, about 90% to about 1000%, about 100% to about 1000%, about 150% to about 1000%, about 200% to about 1000%, about 250% to about 1000%, about 300% to about 1000%, about 350% to about 1000%, about 400% to about 1000%, about 450% to about 1000%, about 500% to about 1000%, about 600% to about 1000%, about 700% to about 1000%, about 800% to about 1000%, about 900% to about 1000%, including all values and ranges in between, compared to the PFS of an otherwise similar patient with cancer that is not treated with Compound I, or a pharmaceutically acceptable salt thereof.

In embodiments, the treatment increases overall survival (OS) compared to OS of an otherwise similar patient with cancer that is not treated with Compound I, or pharmaceutically acceptable salt thereof. In embodiments, the treatment increases OS by at least about 1 week, or about 2 weeks, or about 3 weeks, or about 4 weeks, or about 5 weeks, or about 6 weeks, or about 7 weeks, or about 8 weeks, or about 9 weeks, or about 10 weeks, or about 1 month, or about 2 months, or about 3 months, or about 4 months, or about 5 months, or about 6 months, or about 7 months, or about 8 months, or about 9 months, or about 10 months, or about 11 months, or about 12 months, or about 13 months, or about 14 months, or 15 months, or about 16 months, or about 17 months, or about 18 months, or about 19 months, or about 20 months, or about 21 months, or about 22 months, or about 23 months, or about 24 months, or about 3 years, or about 4 years, or about 5 years, or about 6 years, or about 7 years, or about 8 years, or about 9 years, or about 10 years, including any values and ranges therebetween, compared to OS of an otherwise similar patient with cancer that is not treated with Compound I, or pharmaceutically acceptable salt thereof. In embodiments, the treatment increases OS by at least about 3 months compared to OS of an otherwise similar patient with cancer that is not treated with Compound I, or pharmaceutically acceptable salt thereof. In embodiments, treating according to the methods of the disclosure results in an increase in OS of about 10%, about 20%, about 30%, about 40%, about 50%, about 60%, about 70%, about 80%, about 90%, about 100%, about 150%, about 200%, about 250%, about 300%, about 350%, about 400%, about 450%, about 500%, about 550%, about 600%, about 650%, about 700%, about 750%, about 800%, about 850%, about 900%, about 950%, or about 1000% or more, compared to OS of an otherwise similar patient with cancer that is not treated with Compound I, or pharmaceutically acceptable salt thereof, including all values and ranges in between. In embodiments, the treatment increases OS by about 5% to about 1000%, about 10% to about 1000%, about 20% to about 1000%, about 30% to about 1000%, about 40% to about 1000%, about 50% to about 1000%, about 60% to about 1000%, about 70% to about 1000%, about 80% to about 1000%, about 90% to about 1000%, about 100% to about 1000%, about 150% to about 1000%, about 200% to about 1000%, about 250% to about 1000%, about 300% to about 1000%, about 350% to about 1000%, about 400% to about 1000%, about 450% to about 1000%, about 500% to about 1000%, about 600% to about 1000%, about 700% to about 1000%, about 800% to about 1000%, about 900% to about 1000%, including all values and ranges in between, compared to the PFS of an otherwise similar patient with cancer that is not treated with Compound I, or a pharmaceutically acceptable salt thereof.

### NUMBERED EMBODIMENTS

1. A method of treating advanced or metastatic cancer, comprising administering about 5 mg to about 1500 mg of Compound I having a chemical structure:

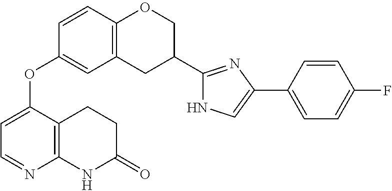

or an equivalent dose of a pharmaceutically acceptable salt or stereoisomer thereof, to a subject in need thereof.

2. The method of embodiment 1, wherein about 10 mg, about 20 mg, about 30 mg, about 40 mg, about 50 mg, about 60 mg, about 75 mg, about 80 mg, about 100 mg, about 150 mg, about 200 mg, about 500 mg, about 800 mg, about 1000 mg, about 1200 mg, or about 1500 mg of Compound I, or an equivalent dose of a pharmaceutically acceptable salt or stereoisomer thereof, is administered.

3. The method of embodiment 1, wherein about 20 mg to about 1000 mg of Compound I, or an equivalent dose of a pharmaceutically acceptable salt or stereoisomer thereof, is administered per day.

4. The method of embodiment 1, wherein about 40 mg to about 1000 mg of Compound I, or an equivalent dose of a pharmaceutically acceptable salt or stereoisomer thereof, is administered per day.

5. The method of embodiment 1, wherein about 1000 mg of Compound I, or an equivalent dose of a pharmaceutically acceptable salt or stereoisomer thereof, is administered per day.

6. The method of any one of embodiments 1-5, wherein Compound I, or an equivalent dose of a pharmaceutically acceptable salt or stereoisomer thereof, is administered once a day or twice a day.

7. The method of embodiment 6, wherein 20 mg of Compound I, or an equivalent dose of a pharmaceutically acceptable salt or stereoisomer thereof, is administered twice a day.

8. The method of embodiment 6, wherein 250 mg of Compound I, or an equivalent dose of a pharmaceutically acceptable salt or stereoisomer thereof, is administered twice a day.

9. The method of embodiment 6, wherein 500 mg of Compound I, or an equivalent dose of a pharmaceutically acceptable salt or stereoisomer thereof, is administered twice a day.

10. The method of any one of embodiments 1-9, wherein Compound I, or an equivalent dose of a pharmaceutically acceptable salt or stereoisomer thereof, is orally administered in a pharmaceutical formulation.

11. The method of embodiment 10, wherein the pharmaceutical formulation is a tablet or a capsule.

12. The method of embodiment 11, wherein the pharmaceutical formulation is a capsule.

13. The method of any one of embodiments 10-12, wherein the pharmaceutical formulation comprises about 5 mg to about 50 mg of Compound I, or an equivalent dose of a pharmaceutically acceptable salt or stereoisomer thereof.

14. The method of any one of embodiments 10-12, wherein the pharmaceutical formulation comprises about 10 mg or about 25 mg of Compound I, or an equivalent dose of a pharmaceutically acceptable salt or stereoisomer thereof.

15. The method of any one of embodiments 1-14, wherein the cancer is Barret's adenocarcinoma, biliary tract carcinomas, breast cancer, cervical cancer, cholangiocarcinoma, central nervous system tumors, primary CNS tumors, glioblastomas, astrocytomas, glioblastoma multiforme, ependymomas, secondary CNS tumors (metastases to the central nervous system of tumors originating outside of the central nervous system), brain tumors, brain metastases, colorectal cancer, large intestinal colon carcinoma, gastric cancer, carcinoma of the head and neck, squamous cell carcinoma of the head and neck, acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma, erythroleukemia, hepatocellular carcinoma, lung cancer, small cell lung cancer, non-small cell lung cancer, ovarian cancer, endometrial cancer, pancreatic cancer, pituitary adenoma, prostate cancer, renal cancer, melanoma, neurofibromatosis type 1 (NF1), neurofibromatosis type 2 (NF2), esophageal cancer, bladder cancer, mesothelioma, or thyroid cancer.

16. The method of embodiment 15, wherein the cancer is non-small cell lung cancer (NSCLC), colorectal cancer, melanoma, or pancreatic cancer.

17. The method of any one of embodiments 1-16, wherein the cancer comprises a solid tumor harboring one or more alterations in the mitogen activated protein kinase (MAPK) pathway.

18. The method of embodiment 17, wherein the one or more alterations is a KRAS mutation, a NRAS mutation, a HRAS mutation, a BRAF mutation, a BRAF fusion, a BRAF translocation, an ARAF mutation, a RAF mutation, an increased receptor tyrosine kinase (RTK) signaling, a RAS gene amplification, a RAF gene amplification, or a combination thereof.

19. The method of embodiment 18, wherein the KRAS mutation is G12C, G12D, G12R, G12V, G12W, G13C, G13D, G13R, G13V, G13W, Q61R, Q61K, Q61L, Q61H, Q61P, A146P, A146T, A146V, A59G, A59T, R68S, H95D, H95Q, H95R, Y96C, or a combination thereof.

20. The method of embodiment 18, wherein the NRAS mutation comprises Q61R, Q61K, Q61L, Q61H, Q61P, G12S, G12D, G12C, G12V, G12A, G12R, G13V, G13R, G13D, G13C, or a combination thereof.

21. The method of embodiment 18, wherein the HRAS mutation comprises Q61R, Q61K, Q61L, G12S, G12D, G12C, G12V, G12A, G12R, G13V, G13R, G13D, G13C, or a combination thereof.

22. The method of embodiment 18, wherein the ARAF mutation comprises G331C, A225V, F351L, P539H, Q347_A348del, N217I, G322S, A451T, R255Gfs(Ter)37, S214(Ter), P8Lfs(Ter)26, G387N/D/R/S, P462L, G377R, or a combination thereof.

23. The method of embodiment 18, wherein the BRAF mutation comprises Class 1 BRAF mutation, Class 2 BRAF mutation, Class 3 BRAF mutation, or a combination thereof.

24. The method of embodiment 18, wherein the BRAF mutation comprises Class 2 or 3 BRAF mutation.

25. The method of embodiment 18, wherein the BRAF mutation comprises V600E, V600M, V600G, V600K, V600R, V600D, G469A, G469V, G469R, K601E, K601N, K601T, L597Q, L597V, G464V, G464E, D594N, D594G, D594A, D594H, G466A, G466V, G466E, N581S, N581I, G469E, S467L, G596D, G596R, F595L, V459L, D287H or a combination thereof.

26. The method of embodiment 18, wherein the RAF mutation comprises S257L/W, S259F, R391S/W, or a combination thereof.

27. The method of embodiment 18, wherein the one or more alterations comprises NRAS Q61K, KRAS G12C, KRAS G13D, KRAS Q61K, or a combination thereof.

28. The method of any one of embodiments 1-27, wherein Compound I is:

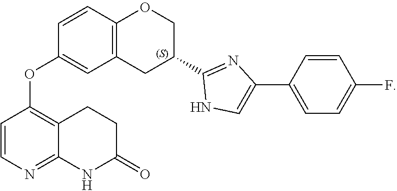

29. The method of embodiment 28, wherein the compound has an enantiomeric excess of at least 85%, at least 90%, at least 95%, or at least 99%.

30. A method of treating cancer harboring one or more alterations in the mitogen activated protein kinase (MAPK) pathway in a subject, comprising:
detecting the one or more alterations in the MAPK pathway; and then
administering about 5 mg to about 1500 mg of Compound I having a chemical structure:

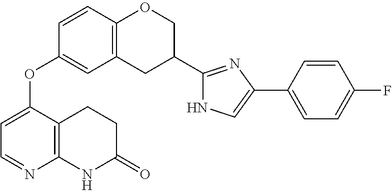

or an equivalent dose of a pharmaceutically acceptable salt or stereoisomer thereof, to the subject.

31. The method of embodiment 30, wherein about 10 mg, about 20 mg, about 40 mg, about 60 mg, about 80 mg, about 100 mg, about 150 mg, about 200 mg, about 500 mg, about 800 mg, about 1000 mg, about 1200 mg, or about 1500 mg of Compound I, or an equivalent dose of a pharmaceutically acceptable salt or stereoisomer thereof, is administered.

32. The method of embodiment 30, wherein about 20 mg to about 1000 mg of Compound I, or an equivalent dose of a pharmaceutically acceptable salt or stereoisomer thereof, is administered per day.

33. The method of embodiment 30, wherein about 40 mg to about 1000 mg of Compound I, or an equivalent dose of a pharmaceutically acceptable salt or stereoisomer thereof, is administered per day 34. The method of embodiment 30, wherein about 1000 mg of Compound I, or an equivalent dose of a pharmaceutically acceptable salt or stereoisomer thereof, is administered per day.

35. The method of embodiment 30, wherein the one or more alterations is a KRAS mutation, a NRAS mutation, a HRAS mutation, a BRAF mutation, a BRAF fusion, a BRAF translocation, an ARAF mutation, a RAF mutation, an increased receptor tyrosine kinase (RTK) signaling, a RAS gene amplification, a RAF gene amplification or a combination thereof.

36. The method of embodiment 35, wherein the KRAS mutation is G12C, G12D, G12R, G12V, G12W, G13C, G13D, G13R, G13V, G13W, Q61R, Q61K, Q61L, Q61H, Q61P, A146P, A146T, A146V, A59G, A59T, R68S, H95D, H95Q, H95R, Y96C, or a combination thereof.

37. The method of embodiment 35, wherein the NRAS mutation comprises Q61R, Q61K, Q61L, Q61H, Q61P, G12S, G12D, G12C, G12V, G12A, G12R, G13V, G13R, G13D, G13C, or a combination thereof.

38. The method of embodiment 35, wherein the HRAS mutation comprises Q61R, Q61K, Q61L, G12S, G12D, G12C, G12V, G12A, G12R, G13V, G13R, G13D, G13C, or a combination thereof.

39. The method of embodiment 35, wherein the ARAF mutation comprises G331C, A225V, F351L, P539H, Q347_A348del, N217I, G322S, A451T, R255Gfs(Ter)37, S214(Ter), P8Lfs(Ter)26, G387N/D/R/S, P462L, G377R, or a combination thereof.

40. The method of embodiment 35, wherein the BRAF mutation comprises Class 1 BRAF mutation, Class 2 BRAF mutation, Class 3 BRAF mutation, or a combination thereof.

41. The method of embodiment 35, wherein the BRAF mutation comprises Class 2 or 3 BRAF mutation.

42. The method of embodiment 35, wherein the BRAF mutation comprises V600E, V600M, V600G, V600K, V600R, V600D, G469A, G469V, G469R, K601E, K601N, K601T, L597Q, L597V, G464V, G464E, D594N, D594G, D594A, D594H, G466A, G466V, G466E, N581S, N581I, G469E, S467L, G596D, G596R, F595L, V459L, D287H or a combination thereof.

43. The method of embodiment 35, wherein the RAF mutation comprises S257L/W, S259F, R391S/W, or a combination thereof.

44. The method of embodiment 30, wherein the one or more alterations comprises NRAS Q61K, KRAS G12C, KRAS G13D, KRAS Q61K, or a combination thereof.

45. The method of any one of embodiments 30-44, wherein the cancer is Barret's adenocarcinoma, biliary tract carcinomas, breast cancer, cervical cancer, cholangiocarcinoma, central nervous system tumors, primary CNS tumors, glioblastomas, astrocytomas, glioblastoma multiforme, ependymomas, secondary CNS tumors (metastases to the central nervous system of tumors originating outside of the central nervous system), brain tumors, brain metastases, colorectal cancer, large intestinal colon carcinoma, gastric cancer, carcinoma of the head and neck, squamous cell carcinoma of the head and neck, acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma, erythroleukemia, hepatocellular carcinoma, lung cancer, small cell lung cancer, non-small cell lung cancer, ovarian cancer, endometrial cancer, pancreatic cancer, pituitary adenoma, prostate cancer, renal cancer, melanoma, neurofibromatosis type 1 (NF1), neurofibromatosis type 2 (NF2), esophageal cancer, bladder cancer, mesothelioma, or thyroid cancer.

46. The method of embodiment 45, wherein the cancer is non-small cell lung cancer (NSCLC), colorectal cancer, melanoma, or pancreatic cancer.

47. The method of any one of embodiments 30-46, wherein Compound I is:

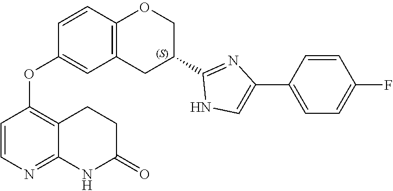

48. The method of embodiment 47, wherein the compound has an enantiomeric excess of at least 85%, at least 90%, at least 95%, or at least 99%.

49. The method of any one of embodiments 30-48, wherein the treatment reduces the tumor size compared to the tumor size prior to administration.

50. The method of embodiment 49, wherein the treatment reduces tumor size by at least about 5%, about 10%, about 20%, about 30%, about 40%, about 50% about 60%, about 70%, about 80%, about 90%, about 95%, or about 100%, compared to the tumor size prior to administration.

51. The method of any one of embodiments 30-48, wherein the treatment increases overall response rate (ORR) compared to the ORR of an otherwise similar patient with cancer that is not treated with Compound I.

52. The method of embodiment 51, wherein the treatment increases ORR by at least about 5%.

53. The method of embodiment 52, wherein the treatment increases ORR by 10% to about 100%.

54. The method of any one of embodiments 30-48, wherein the treatment increases progression free survival (PFS) compared to the PFS of an otherwise similar patient with cancer that is not treated with Compound I.

55. The method of embodiment 54, wherein the treatment increases PFS by at least about 3 months.

56. The method of embodiment 55, wherein the treating increases PFS by at least about 6 months, 12 months, at least about 18 months, or at least about 24 months.

57. The method of any one of embodiments 30-48, wherein the treatment increases overall survival (OS) compared to the OS of an otherwise similar patient with cancer that is not treated with Compound I.

58. The method of embodiment 57, wherein the treatment increases OS by at least about 3 months.

### Examples

### Example 1. Compound I is a Potent Pan-RAF Inhibitor

To evaluate the ability of Compound I to inhibit different RAF kinases, IC₅₀ values of Compound I were measured using two different biochemical cell-free kinase assays: RBC radiometric HotSpot™ filter-based kinase assay (Reaction Biology, reaction components include 15-25 μM ATP, 3-200 nM recombinant kinase, 5 μM kinase dead MEK1 K97R substrate) and PanQinase™ flash plate-based kinase assay (Reaction Biology, 0.25-0.5 μg/50 μL kinase dead MEK K97M substrate and 0.1-3 μM of ATP) following manufacturer's protocols.

Compound I's inhibitory effect against wildtype ARAF, BRAF, and CRAF kinase isoforms and specifically designed mutant forms of ARAF (P462L, G377R, G387D, del 347_348, and del 347-348+G387D), BRAF (V600E, G469A, Fusion-FAM131B), and CRAF (Dimer-RAF1 and R391W) were tested. The results demonstrate that Compound I is a pan-inhibitor of all three RAF isoforms and selected mutants at a nanomolar IC₅₀ range. The IC₅₀ values are summarized in Table 1 (measured by using HopSpot filter-based assay platform) and Table 2 (average IC₅₀ measured by using PanQinase™ flash plate-based assay platform).

TABLE 1
IC₅₀ for Compound I

| Kinase | Sequence | IC₅₀ (nM) |
|---|---|---|
| ARAF* | WT | 0.33 |
| BRAF | WT | 1.8 |
| | V600E (Class 1) | 0.46 |
| | G469A (Class 2) | 1.4 |
| | Fusion (FAM131B) | 1.5 |
| CRAF* | CRAF/RAF1 | 0.3 |
| | R391W | 0.29 |

*All kinase domain assays with ARAF and CRAF contain Y340D/Y341D

TABLE 2
IC₅₀ for Compound I

| Kinase | Sequence | IC₅₀ (nM) |
|---|---|---|
| ARAF* | WT | 15.7 |
| | P462L | 117.2 |
| | G377R | 141.7 |
| | del 347_348 | 68.3 |
| | Del 347_348 G387D | 20.4 |
| BRAF | WT | 40.6 |
| | V600E | 40.2 |
| CRAF* | WT | 2.3 |

*All kinase domain assays with ARAF and CRAF contain Y340D/Y341D

To assess the potency and selectivity of Compound I against different RAF mutants, the average IC₅₀ values of Compound I for RAF kinase isoforms and their mutants were measured and further compared with the average IC₅₀ of other known pan-inhibitors, belvarafenib and naporafenib, by using PanQinase™ flash plate-based kinase assay with 0.25-0.5 μg/50 μL kinase dead MEK K97M substrate and 0.1-3 μM of ATP. The IC₅₀ values are summarized in Table 3. As shown in Table 3, Compound I is a significantly more potent inhibitor (3- to 5-fold lower IC₅₀ values) than belvarafenib or naporafenib for all three RAF isoforms, including their mutants, particularly, ARAF P462L and ARAF G377R mutant kinases.

TABLE 3
IC₅₀ for Compound I, belvarafenib, and naporafenib

| | | IC₅₀, nM | | |
|---|---|---|---|---|
| Kinase | Sequence | JZP815 | Belvarafenib | Naporafenib |
| ARAF | WT | 11.4 | 26.0 | 63.5 |
| | P462L | 117.2 | 361.5 | 401.1 |
| | G377R | 141.7 | 753.5 | 621.4 |
| | del 347_348 | 68.3 | 60.3 | 153.9 |
| | del 347_348 | 20.4 | 46.5 | 67.2 |
| | G387D | | | |
| BRAF | WT | 40.6 | 97.2 | 141.5 |
| | V600E | 40.2 | 193.2 | 191.0 |
| CRAF | WT | 2.3 | 9.1 | 9.7 |

### Example 2. Preclinical Studies for Determining Doses of Compound I

Compound I was evaluated in GLP-compliant 28-day oral toxicity studies in 2 preclinical species, Sprague Dawley rats and beagle dogs. In the rat 28-day study, Compound I was administered at 0, 10, 20, or 45 mg/kg/day once daily via oral gavage. No mortality, and no clinical signs, changes in body weight or food consumption, ophthalmology, clinical pathology or macroscopic findings of toxicological relevance were noted. In the dog 28-day study, Compound I was administered at 0, 3.75, 7.5, or 15 mg/kg/day once daily via oral gavage. No mortality, and no clinical signs, changes in body weight or food consumption, ophthalmology, ECG, clinical pathology, macroscopic or microscopic findings of toxicological relevance were noted.

Using PK/PD/efficacy simulations, an efficacious human dose (500 mg/dose BID) and human PK parameters (allometrically scaled CL of 1.2 mL/min/kg, Vss of 0.77 L/kg, and assumed oral % F approximately 40%, and Compound I unbound plasma Ctau of approximately 15 nM) were predicted.

### Example 3. Preclinical Efficacy Assessment of Compound I in Tumor Xenograft Mice Models

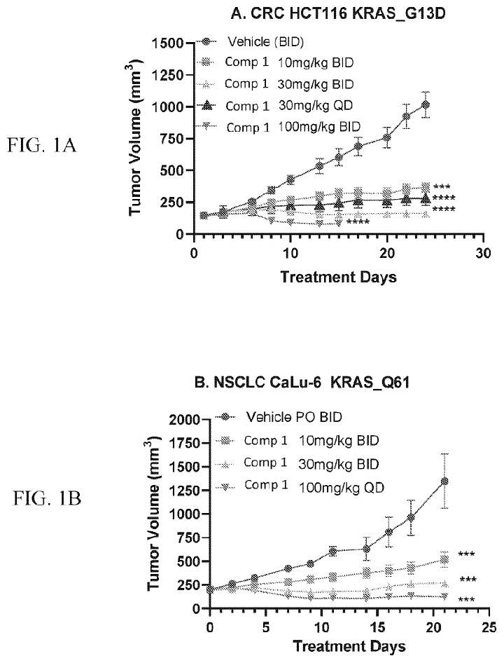

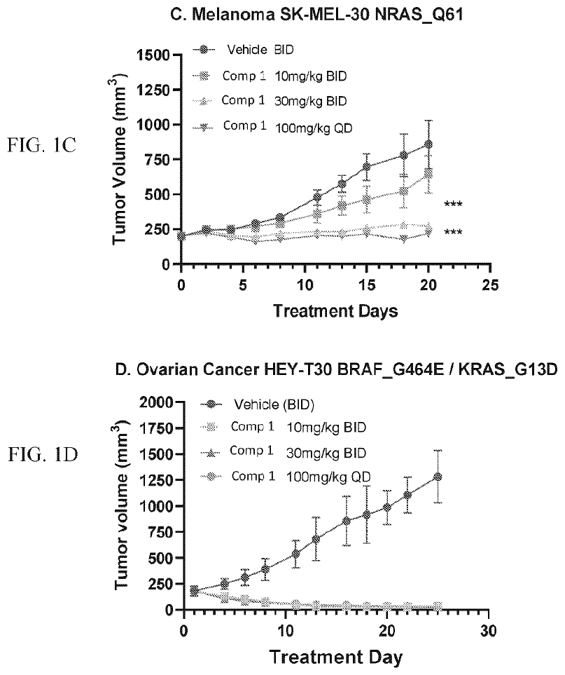

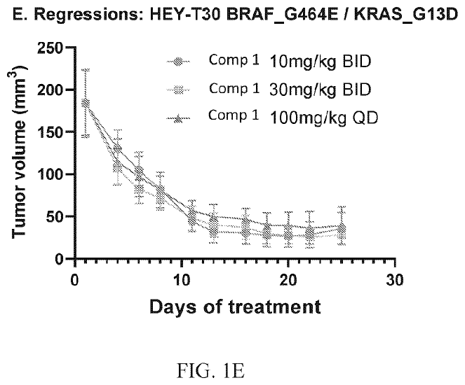

The effect of Compound I in tumor growth inhibition was evaluated in xenograft mice models carrying pathogenic RAS (NRAS and KRAS) and RAF (BRAF Class 2/3) mutation driven colorectal cancer, non-small cell lung cancer, melanoma, and ovarian cancer. A dose-dependent tumor growth inhibition effect of Compound I was observed as shown in FIGS. 1A-E.

### Example 4. a Phase 1 Study of Compound I Oral Capsules in Adult Participants with Advanced or Metastatic Solid Tumors Harboring Mitogen Activated Protein Kinase (MAPK) Pathway Alterations to Investigate the Safety, Dosing, and Antitumor Activity of Compound I

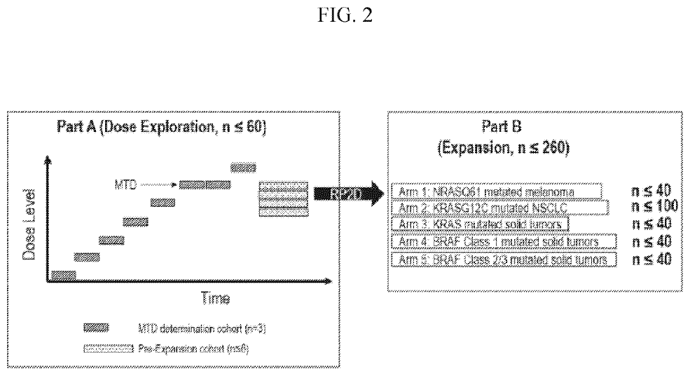

This is a phase 1 study of Compound I in participants with advanced or metastatic solid tumors harboring alterations in the MAPK pathway. The study comprises 2 parts: dose exploration (Part A) and expansion (Part B). The study design is summarized in FIG. 2.

### Part A

Primary objectives (Part A): To determine the recommended phase 2 dose (RP2D) and/or a maximum tolerance dose (MTD) of Compound I that may be safely administered to participants with advanced and/or metastatic solid tumors harboring an alteration in the MAPK pathway; and to characterize the safety profile of Compound I.

Secondary objectives (Part A): To characterize plasma PK of Compound I and its metabolites; to characterize the dose proportionality of Compound I and its metabolites; and to assess antitumor activity.

Exploratory objectives (Part A): To explore pharmacodynamics and ctDNA biomarkers; to explore the effect of Compound I on QTc and on other ECG parameters following oral doses of Compound I; to characterize urinary PK of Compound I, and its metabolites (if applicable); to perform a preliminary Compound I metabolite identification in plasma and urine samples; to assess antitumor activity; to assess duration of response (DoR) and progression-free survival (PFS); and to explore the effect of food on Compound I plasma PK.

### Endpoints (Part A):

a. Incidence and nature of dose-limiting toxicity (DLTs) (Part A maximum tolerance dose (MTD) determination cohorts only)
b. Incidence and severity of adverse events (AEs) and serious adverse events (SAEs), changes in laboratory values, vital signs, and other safety measures
c. Dose interruptions and reductions
d. Compound I and its metabolites plasma PK parameters (including, but not limited to, Cmax, Tmax, AUC∞, AUCtau, t1/2, CL/F (Compound I only), Vz/F (Compound I only), accumulation ratio, metabolite-to-parent ratio, if applicable)
e. Compound I and its metabolites plasma concentrations
f. Dose proportionality of Compound I and its metabolites as determined by the slopes and associated 90% CIs for Cmax and AUC
g. Objective response rate (ORR), defined as confirmed complete response (CR)+partial response (PR), by RECIST v1.1
h. Changes from baseline of intratumor and/or peripheral blood mononuclear cell MAPK signaling and ctDNA levels
i. Corrected QT interval (QTc) interval and change from baseline in QTc interval
j. PR interval and change from baseline in PR interval
k. QRS interval and change from baseline in QRS interval
l. Heart rate and change from baseline in heart rate
m. Compound I and its metabolites (if applicable) urine PK parameters (including but not limited to amount excreted (Ae), renal clearance (CLr), if applicable)
n. Preliminary structures of Compound I metabolites in plasma and urine
o. The disease control rate, defined as best overall response (BOR) of stable disease (SD)+complete response (CR)+partial response (PR)
p. Duration of Response (DoR)
q. Progression free survival (PFS)
r. Effect of food on Compound I plasma PK as determined by the 90% CIs for plasma Cmax, ss, AUCtau in fed versus fasting state

### Part B

Primary objectives (Part B): To further assess antitumor activity of Compound I at the RP2D in different tumor/mutation types; and to further characterize the safety profile of Compound I at the RP2D.

Secondary objectives (Part B): To assess PFS and OS.

Exploratory objectives (Part B): To assess antitumor activity; to explore PD and ctDNA, biomarkers; and to characterize plasma PK of Compound I, and its metabolites (if applicable).

### Endpoints (Part B):

a. ORR, defined as confirmed CR+PR, by RECIST v1.1
b. DoR
c. Incidence and severity of AEs and SAEs, changes in laboratory values, vital signs, and other safety measures
d. Dose interruptions and reductions
e. PFS and OS
f. The disease control rate, defined as BOR of SD+CR+PR
g. Changes from baseline of intratumor and/or peripheral blood mononuclear cell MAPK signaling and ctDNA levels
h. Compound I and its metabolites (if applicable) plasma concentrations

The study comprises 3 periods: (i) screening period (up to 28 days before the treatment period); (ii) treatment period (the length of treatment cycles is 28 days); and (iii) follow-up period (Safety Follow-up Visit 30 days (±3 days) after last dose of Compound I). Participant will be followed up for survival by telephone call every 12 weeks ±1 week from the End of treatment until death, withdrawal of consent, or lost to follow-up.

### Inclusion Criteria

Participants are eligible to be included in the study only if all of the following criteria apply:
a. Participant must be ≥18 years of age, at the time of signing the informed consent.
b. Participants who have histological or cytological diagnosis of an advanced or metastatic solid tumor carrying a documented, clinically significant, MAPK pathway alteration. Participants must have exhausted all available standard of care therapies, or in the opinion of the investigator would be unlikely to tolerate or derive clinically meaningful benefit from available standard of care therapy.
c. Performance status (ECOG) of 0 or 1, measured within 72 hours before start of treatment
d. have measurable disease by RECIST v1.1
e. Tumor must be safely amenable to core needle or excisional biopsy.
f. Renal: creatinine clearance of ≥60 mL/min; Hepatic: total bilirubin≤1.5× ULN; AST and ALT≤2.5×ULN or ≤5×ULN if due to tumor involvement in liver; Bone marrow: hemoglobin ≥9.0 g/dL; platelets ≥100×10⁹ cells/L; absolute neutrophil count ≥1.5×10⁹ cells/L).
g. Expected life expectancy of at least 12 weeks
h. For each arm in Part B (Expansion), participant must be diagnosed with the tumor type(s) carrying the mutation(s) specified. For Arm 1 (NRASQ61 mutated melanoma), participants must have received prior immunotherapy (unless contraindicated). For Arm 2 (KRASG12C mutated NSCLC), participants must have received prior platinum-based combination therapy and/or (if applicable) targeted therapy for mutations in EGFR, KRAS, ALK or ROS1, or PD-L1 expression (unless contraindicated).
i. Male and Female must agree to use contraception.

### Exclusion Criteria:

a. Known uncontrolled brain metastases. Stable brain metastases either treated or being treated with a stable dose of steroids/anticonvulsants, with no dose change in the previous 4 weeks, are permitted.
b. Active fungal, bacterial and/or known viral infection including HIV or Hepatitis A, B, C
c. Concomitant malignancies or previous malignancies with less than 2 years disease-free interval at the time of enrollment, with the exception of non-metastatic, non-melanomatous skin cancers, carcinoma in-situ, melanoma in-situ, prostate cancer with undetectable PSA that are adequately treated. Other concurrent malignancies that are indolent and do not require active treatment must be discussed with the medical monitor prior to enrollment.
d. Has clinically significant (ie, active) cardiovascular disease: cerebral vascular accident/stroke (<6 months prior to enrollment), myocardial infarction (<6 months prior to enrollment), unstable angina, congestive heart failure (>New York Heart Association Classification Class II), QTc ≥470 msec, or serious cardiac arrhythmia requiring medication.
e. Uncontrolled or severe intercurrent medical condition.
f. Gastrointestinal condition that could impair absorption of study intervention (specific cases may be enrolled after discussion with the sponsor), or inability to ingest study intervention.
g. Received any cancer directed therapy (chemotherapy, hormonal therapy, biologic, etc.) within 28 days or 5 half-lives (whichever is shorter) of starting study intervention. Participants who have received radiotherapy must have recovered from acute toxicities associated with treatment.
h. Use of any products or medicines known to be strong or moderate inducers or inhibitors of CYP3A4, which cannot be discontinued at least 4 weeks.
i. Use of proton pump inhibitors and histamine-2 receptor antagonists, which cannot be discontinued at least 2 weeks before first dose.
j. Use of concurrent therapy.

### Designs:

### Study Part A

The maximum tolerance dose (MTD) in participants with solid tumors harboring MAPK alterations will be determined based on the incidence of dose-limiting toxicity (DLTs). The DLT observation period will be Cycle 1 (28 days). DLT determination will be based on a regimen in which participants receive Compound I daily. Based on the safety data/DLTs and PK data, the safety review committee (SRC) may determine that no additional dose escalations should be investigated and/or that determination of the MTD should stop. The starting dose level (Dose Level 1) of Compound I is 20 mg BID. After at least 3 evaluable participants are treated at Dose Level 1, dose exploration investigating up to 5 additional dose levels described herein.

### Study Part B

Part B involves multiple nonrandomized expansion arms, defined by MAPK alteration and/or tumor type, that will be treated at the recommended phase 2 dose (RP2D).

The arms for Part B will be as follows:
Arm 1: NRAS Q61 mutated melanoma
Arm 2: KRAS G12C mutated NSCLC
Arm 3: KRAS mutated solid tumors
Arm 4: BRAF Class 1 mutated solid tumors
Arm 5: BRAF Class 2/3 mutated solid tumors

Exemplary MAPK alterations are listed in Table 4.

TABLE 4
MAPK Alterations

| Gene | Alteration | |
|---|---|---|
| KRAS | mutation | G12C/D/R/V/W; G13C/D/R/V/W; Q61R/K/L/H/P; A146P/T/V; |
| | | A59G/T; R68S; H95D/Q/R; Y96C |
| NRAS | mutation | Q61R/K/L/H/P; G12S/D/C/V/A/R; G13V/R/D/C |
| HRAS | mutation | Q61R/K/L; G12S/D/C/V/A/R; G13V/R/D/C |
| BRAF | Class 1 | V600E/M/G/K/R/D |
| | Class 2 | G469A/V/R; K601E/N/T; L597Q/V; G464V/E |
| | Class 3 | D594N/G/A/H; G466A/V/E; N581S/I; G469E; S467L; G596D/R; |
| | | F595L; V459L; D287H |
| BRAF | Fusion | Multiple |
| | (Class 2) | |
| BRAF | translocation | Multiple |
| ARAF | mutation | G331C; A225V; F351L; P539H; Q347_A348del; N217I; G322S; |
| | | A451T; R255Gfs(Ter)37; S214(Ter); P8Lfs(Ter)26; G387N/D/R/S; |
| | | P462L; G377R |
| RAF1 | mutation | S257L/W; S259F; R391S/W |
| EGFR | mutation | L858R; G719C/A/D; L861Q; A289V/D/T; E709K; S768I; |
| | | G598V/E/A; L833V/F; R108K/G; R252C; T790M; C797S; exon 19 |
| | | deletions |

### Schedule of Activities

| | | | | | | | | | | | | End of | Follow-up |
|---|---|---|---|---|---|---|---|---|---|---|---|---|---|
| | | Treatment | | | | | | | | | | Treatment | Safety |
| | | | | | | | | | | | Cycle 3 | Visit | Follow-up |
| | Screening | Cycle 1 | | | | | | | Cycle 2 | | to n | 0 to 21 d | Visit 30 d |
| | Relative Day Within a Cycle | | | | | | | | | | | after last | after last |
| Procedure | −28 to −1 | 1 ± 0 | 2 ± 0 | 3 ± 0 | 4 ± 0 | 8 ± 1 | 15 ± 1 | 22 ± 1 | 1 ± 1 | 15 ± 1 | 1 ± 2 | dose | dose ±3 |
| Informed consent | X | — | — | — | — | — | — | — | — | — | — | — | — |
| Review inclusion and | X | X | — | — | — | — | — | — | — | — | — | — | — |
| exclusion criteria | | | | | | | | | | | | | |
| Demography | X | — | — | — | — | — | — | — | — | — | — | — | — |
| Medical/disease | X | — | — | — | — | — | — | — | — | — | — | — | — |
| history | | | | | | | | | | | | | |
| Tumor mutation | X | — | — | — | — | — | — | — | — | — | — | — | — |
| testing | | | | | | | | | | | | | |
| Treatment history | X | — | — | — | — | — | — | — | — | — | — | — | — |
| and prior | | | | | | | | | | | | | |
| medications | | | | | | | | | | | | | |
| Physical examination | X | X | — | — | — | — | X | — | X | X | X | X | — |
| Weight | X | X | — | — | — | — | — | — | X | — | X | X | — |
| Height | X | — | — | — | — | — | — | — | — | — | — | — | — |
| ECOG performance | X | X | — | — | — | — | — | — | X | — | X | X | — |
| status | | | | | | | | | | | | | |
| Vital signs | X | X | — | — | — | X | X | X | X | X | X | X | — |
| Dermatologic | X | — | — | — | — | — | — | — | — | — | X | X | — |
| examination | | | | | | | | | | | | | |
| 12-lead ECG | X | X | — | — | — | — | X | — | — | — | X | X | — |
| (Part A) | | | | | | | | | | | | | |
| 12-lead ECG | X | — | — | — | — | — | — | — | — | — | X | X | — |
| (Part B) | | | | | | | | | | | | | |
| Holter | — | X | — | — | — | | X | — | — | — | — | — | — |
| monitoring (Part | | | | | | | | | | | | | |
| A only) | | | | | | | | | | | | | |
| Serum or urine | X | X | — | — | — | — | — | — | X | — | X | X | X |
| pregnancy test | | | | | | | | | | | | | |
| (WOCBP only) | | | | | | | | | | | | | |
| Hematology | X | X | — | — | — | X | X | X | X | X | X | X | — |
| Chemistry | X | X | — | — | — | X | X | X | X | X | X | X | — |
| aPTT, PT, INR | X | — | — | — | — | — | — | — | — | — | — | X | — |
| Urinalysis | X | X | — | — | — | — | — | — | X | — | X | — | — |
| Blood sample - | X | — | — | — | — | — | — | — | X | — | X | X | — |
| banking | | | | | | | | | | | | | |
| Tumor banking | X | — | — | — | — | — | — | — | — | — | — | — | — |
| Tumor measurement | X | — | — | — | — | — | — | — | — | — | X | X | — |
| (palpable or | | | | | | | | | | | | | |
| measurable) | | | | | | | | | | | | | |
| Radiological tumor | X | — | — | — | — | — | — | — | — | — | X | X | — |
| assessment (as per | | | | | | | | | | | | | |
| RECIST v1.1) | | | | | | | | | | | | | |
| Tumor biopsy for | X | — | — | — | — | — | X | — | — | — | — | — | — |
| pharmacodynamics | | | | | | | | | | | | | |
| Blood sample for | X | — | — | — | — | — | X | — | — | — | — | — | — |
| pharmacodynamics (Part A | | | | | | | | | | | | | |
| only) | | | | | | | | | | | | | |
| Intensive plasma PK | — | X | X | X | X | — | X | — | — | — | — | — | — |
| sampling (Part A MTD | | | | | | | | | | | | | |
| determination cohorts for | | | | | | | | | | | | | |
| Dose Levels 1 and 2) | | | | | | | | | | | | | |
| Intensive plasma PK | — | X | — | — | — | — | X | — | — | — | — | — | — |
| sampling (Part A MTD | | | | | | | | | | | | | |
| determination cohorts for | | | | | | | | | | | | | |
| Dose Levels > 2 and | | | | | | | | | | | | | |
| Pre-Expansion cohorts | | | | | | | | | | | | | |
| other than the Pre- | | | | | | | | | | | | | |
| Expansion food effect | | | | | | | | | | | | | |
| cohort) | | | | | | | | | | | | | |
| Intensive plasma PK | — | X | — | — | — | X | X | — | — | — | — | — | — |
| sampling (Part A Pre- | | | | | | | | | | | | | |
| Expansion food effect | | | | | | | | | | | | | |
| cohort) | | | | | | | | | | | | | |
| Sparse plasma PK sampling | — | — | — | — | — | — | — | — | — | X | X | — | — |
| (Part A) | | | | | | | | | | | | | |

| | | | | | | | | | | | | End of | Follow-up |
|---|---|---|---|---|---|---|---|---|---|---|---|---|---|
| | | Treatment | | | | | | | | | | Treatment | Safety |
| | | | | | | | | | | | Cycle 3 | Visit | Follow-up |
| | Screening | Cycle 1 | | | | | | | Cycle 2 | | to n | 0 to 21 d | Visit 30 d |
| | Relative Day Within a Cycle | | | | | | | | | | | after last | after last |
| Procedure | −28 to −1 | 1 ± 0 | 2 ± 0 | 3 ± 0 | 4 ± 0 | 8 ± 1 | 15 ± 1 | 22 ± 1 | 1 ± 1 | 15 ± 1 | 1 ± 2 | dose | dose ±3 |
| Sparse plasma PK | — | — | — | — | — | X | — | X | — | X | X | — | — |
| sampling (Part B) | | | | | | | | | | | | | |
| Urine PK sampling | — | X | — | — | — | — | — | — | — | — | — | — | — |
| (Part A | | | | | | | | | | | | | |
| Pre-Expansion cohort | | | | | | | | | | | | | |
| dosed at MTD or | | | | | | | | | | | | | |
| highest dose) | | | | | | | | | | | | | |
| High-fat, high- | — | — | — | — | — | X | — | — | — | — | — | — | — |
| calorie meal (Part A | | | | | | | | | | | | | |
| Pre-Expansion food | | | | | | | | | | | | | |
| effect cohort) | | | | | | | | | | | | | |
| Dispense Compound I | — | X | — | — | — | — | — | — | X | — | X | — | — |
| Administer | — | X | | | | | | | X | | X | — | — |
| Compound I | | | | | | | | | | | | | |

| | |
|---|---|
| Survival | Participants will be followed for survival by telephone call every |
| Follow-up | 12 weeks ± 1 week from the End of Treatment until death, |
| | withdrawal of consent, or lost to follow-up until 18 months after |
| | the last participant starts study intervention. Antineoplastic |
| | therapy that the participant is receiving should be collected |
| | during survival follow-up. |

Compound I (i.e., (S)-5-((3-(4-(4-fluorophenyl)-1H-imidazol-2-yl) chroman-6-yl)oxy)-3,4-dihydro-1,8-naphthyridin-2 (1H)-one) is orally administered to participants BID in a capsule formulation with unit dose strengths of 10 mg or 25 mg, taken approximately 12 hours apart in the morning and in the evening. Specifically, in Part A, Compound I is administered as a single dose on Cycle 1 Day 1, with no doses administered on Days 2 and 3 before proceeding with BID dosing on Day 4 and after. Based on the PK data, QD dosing is also being investigated in Part A where Compound I is administered as a single dose on Cycle 1 Day 1, with no doses administered on Days 2 and 3 before proceeding with QD dosing on Day 4 and after.

### Efficacy Assessments

Tumor response to therapy will be assessed by the investigator using RECIST v1.1. Tumor assessments will be made by CT/MRI/PE at Screening, on Day 1 of Cycle 3 (Day 57), and every 2 cycles (56 days) thereafter until Dayl of Cycle 13, after which tumor assessments will be every 3 cycles (84 days) and at the End of Treatment Visit. Tumor assessments will include all known or suspected disease sites. Imaging must include chest, abdomen, and pelvis CT or MRI scans. Imaging will be performed on the abdomen, chest, pelvis, and the site of the primary tumor if it is located elsewhere.

Safety will be assessed by performing the followings: physical Examinations, vital signs, electrocardiograms, dermatologic examination, and clinical safety laboratory assessments.

### Example 5. Safety and Efficacy Assessment of Compound I in Human Cancer Patients

Dosing regimens: To assess the safety and efficacy of Compound I in the cancer treatment, Compound I was orally administered to participants at 20 mg BID (“bis in die”, or “twice a day”), 30 mg BID, 50 mg BID, 50 mg QD (“quaque die”, “once a day”), 75 mg BID, or 100 mg QD for a period of time. Treatment-related adverse events (TRAE, Grade≤2 and Grade ≥3) were monitored and recorded. The efficacy of Compound I in treating cancer was determined by using radiologic tumor assessment as per RECIST v1.1 (e.g., PET-CT or MRI scans).

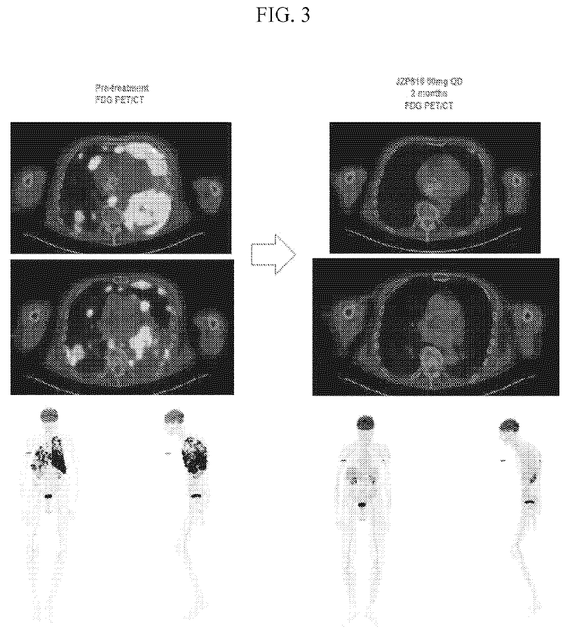

Result: Participant (n=1) with metastatic NRASQ61 mutated melanoma achieved a partial response (PR) with a 61% reduction in target lesions after 2 months and a confirmation of this PR with a 69% reduction in the target lesion after 4 months on a dose of 50 mg QD of Compound I, along with significant resolution of PET avidity (FIG. 3).

Additional dosing regimens of Compound will be investigated (summarized in Table 5).

TABLE 5
Additional dosing regimens of Compound I
Compound I dosing regimens

| | | | | |
|---|---|---|---|---|
| 5 mg QD | 75 mg QD | 140 mg QD | 225 mg BID (450 | 375 mg QD |
| | | | mg total daily | |
| | | | dose) | |
| 5 mg BID (10 | 80 mg BID (160 | 150 mg BID (300 | 225 mg QD | 400 mg BID (800 |
| mg total daily | mg total daily | mg total daily | | mg total daily |
| dose) | dose) | dose) | | dose) |
| 20 mg QD | 80 mg QD | 150 mg QD | 275 mg BID (550 | 400 mg QD |
| | | | mg total daily | |
| | | | dose) | |
| 25 mg QD | 90 mg BID (180 | 160 mg BID (320 | 275 mg QD | 425 mg BID (850 |
| | mg total daily | mg total daily | | mg total daily |
| | dose) | dose) | | dose) |
| 25 mg BID (50 | 90 mg QD | 160 mg QD | 300 mg BID (600 | 425 mg QD |
| mg total daily | | | mg total daily | |
| dose) | | | dose) | |
| 30 mg QD | 100 mg BID (200 | 175 mg BID (350 | 300 mg QD | 450 mg BID (900 |
| | mg total daily | mg total daily | | mg total daily |
| | dose) | dose) | | dose) |
| 40 mg QD | 120 mg BID (240 | 175 mg QD | 325 mg BID (650 | 450 mg QD |
| | mg total daily | | mg total daily | |
| | dose) | | dose) | |
| 60 mg BID (120 | 120 mg QD | 180 mg BID (360 | 325 mg QD | 475 mg BID (950 |
| mg total daily | | mg total daily | | mg total daily |
| dose) | | dose) | | dose) |
| 60 mg QD | 130 mg BID (260 | 180 mg QD | 350 mg BID (700 | 475 mg QD |
| | mg total daily | | mg total daily | |
| | dose) | | dose) | |
| 70 mg BID (140 | 130 mg QD | 200 mg BID (400 | 350 mg QD | 500 mg BID |
| mg total daily | | mg total daily | | (1000 mg total |
| dose) | | dose) | | daily dose) |
| 70 mg QD | 140 mg BID (280 | 200 mg QD | 375 mg BID (750 | 500 mg QD |
| | mg total daily | | mg total daily | |
| | dose) | | dose) | |

1. A method of treating advanced or metastatic cancer, comprising administering about 5 mg to about 500 mg of Compound I: 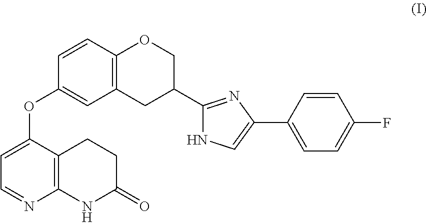
or an equivalent dose of a pharmaceutically acceptable salt thereof, to a subject in need thereof.

2. The method of claim 1, wherein about 5 mg, about 10 mg, about 20 mg, about 25 mg, about 30 mg, about 40 mg, about 50 mg, about 60 mg, about 75 mg, about 80 mg, about 100 mg, about 150 mg, about 175 mg, about 200 mg, about 225 mg, about 275 mg, about 325 mg, about 375 mg, about 425 mg, about 475 mg, or about 500 mg of Compound I, or an equivalent dose of a pharmaceutically acceptable salt thereof, is administered.

3. The method of claim 1, wherein about 20 mg to about 500 mg of Compound I, or an equivalent dose of a pharmaceutically acceptable salt or thereof, is administered per day.

4. The method of claim 1, wherein about 20 mg to about 300 mg of Compound I, or an equivalent dose of a pharmaceutically acceptable salt thereof, is administered per day.

5. The method of claim 1, wherein about 20 mg to about 200 mg of Compound I, or an equivalent dose of a pharmaceutically acceptable salt thereof, is administered per day.

6. The method of claim 1, wherein Compound I, or an equivalent dose of a pharmaceutically acceptable salt thereof, is administered once a day or twice a day.

7. The method of claim 6, wherein about 20 mg of Compound I, or an equivalent dose of a pharmaceutically acceptable salt thereof, is administered twice a day.

8. The method of claim 6, wherein about 30 mg of Compound I, or an equivalent dose of a pharmaceutically acceptable salt thereof, is administered twice a day.

9. The method of claim 6, wherein about 50 mg of Compound I, or an equivalent dose of a pharmaceutically acceptable salt thereof, is administered once a day.

10. The method of claim 6, wherein about 50 mg of Compound I, or an equivalent dose of a pharmaceutically acceptable salt thereof, is administered twice a day.

11. The method of claim 6, wherein about 75 mg of Compound I, or an equivalent dose of a pharmaceutically acceptable salt thereof, is administered twice a day.

12. The method of claim 6, wherein about 100 mg of Compound I, or an equivalent dose of a pharmaceutically acceptable salt thereof, is administered once a day.

13. The method of claim 6, wherein about 250 mg of Compound I, or an equivalent dose of a pharmaceutically acceptable salt thereof, is administered twice a day.

14. The method of claim 6, wherein about 500 mg of Compound I, or an equivalent dose of a pharmaceutically acceptable salt thereof, is administered twice a day.

15. The method of any one of claims 1-14, wherein Compound I, or an equivalent dose of a pharmaceutically acceptable salt thereof, is orally administered in a pharmaceutical formulation.

16. The method of claim 15, wherein the pharmaceutical formulation is a tablet or a capsule.

17. The method of claim 16, wherein the pharmaceutical formulation is a capsule.

18. The method of any one of claims 15-17, wherein the pharmaceutical formulation comprises about 5 mg to about 50 mg of Compound I, or an equivalent dose of a pharmaceutically acceptable salt thereof.

19. The method of any one of claims 15-17, wherein the pharmaceutical formulation comprises about 10 mg or about 25 mg of Compound I, or an equivalent dose of a pharmaceutically acceptable salt thereof.

20. The method of any one of claims 1-19, wherein the cancer is Barret's adenocarcinoma, biliary tract carcinomas, breast cancer, cervical cancer, cholangiocarcinoma, central nervous system tumors, primary CNS tumors, glioblastomas, astrocytomas, glioblastoma multiforme, ependymomas, secondary CNS tumors (metastases to the central nervous system of tumors originating outside of the central nervous system), brain tumors, brain metastases, colorectal cancer, large intestinal colon carcinoma, gastric cancer, carcinoma of the head and neck, squamous cell carcinoma of the head and neck, acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma, erythroleukemia, hepatocellular carcinoma, lung cancer, small cell lung cancer, non-small cell lung cancer, ovarian cancer, endometrial cancer, pancreatic cancer, pituitary adenoma, prostate cancer, renal cancer, melanoma, neurofibromatosis type 1 (NF1), neurofibromatosis type 2 (NF2), esophageal cancer, bladder cancer, mesothelioma, or thyroid cancer.

21. The method of claim 20, wherein the cancer is non-small cell lung cancer (NSCLC), colorectal cancer, melanoma, or pancreatic cancer.

22. The method of any one of claims 1-21, wherein the cancer comprises a solid tumor harboring one or more alterations in the mitogen activated protein kinase (MAPK) pathway.

23. The method of claim 22, wherein the one or more alterations is a KRAS mutation, a NRAS mutation, a HRAS mutation, a BRAF mutation, a BRAF fusion, a BRAF translocation, an ARAF mutation, a RAF mutation, an increased receptor tyrosine kinase (RTK) signaling, a RAS gene amplification, a RAF gene amplification or a combination thereof.

24. The method of claim 23, wherein the KRAS mutation is G12C, G12D, G12R, G12V, G12W, G13C, G13D, G13R, G13V, G13W, Q61R, Q61K, Q61L, Q61H, Q61P, A146P, A146T, A146V, A59G, A59T, R68S, H95D, H95Q, H95R, Y96C, or a combination thereof.

25. The method of claim 23, wherein the NRAS mutation comprises Q61R, Q61K, Q61L, Q61H, Q61P, G12S, G12D, G12C, G12V, G12A, G12R, G13V, G13R, G13D, G13C, or a combination thereof.

26. The method of claim 23, wherein the HRAS mutation comprises Q61R, Q61K, Q61L, G12S, G12D, G12C, G12V, G12A, G12R, G13V, G13R, G13D, G13C, or a combination thereof.

27. The method of claim 23, wherein the ARAF mutation comprises G331C, A225V, F351L, P539H, Q347_A348del, N217I, G322S, A451T, R255Gfs(Ter)37, S214(Ter), P8Lfs(Ter)26, G387N/D/R/S, P462L, G377R, or a combination thereof.

28. The method of claim 23, wherein the BRAF mutation comprises Class 1 BRAF mutation, Class 2 BRAF mutation, Class 3 BRAF mutation, or a combination thereof.

29. The method of claim 23, wherein the BRAF mutation comprises Class 2 or 3 BRAF mutation.

30. The method of claim 23, wherein the BRAF mutation comprises V600E, V600M, V600G, V600K, V600R, V600D, G469A, G469V, G469R, K601E, K601N, K601T, L597Q, L597V, G464V, G464E, D594N, D594G, D594A, D594H, G466A, G466V, G466E, N581S, N581I, G469E, S467L, G596D, G596R, F595L, V459L, D287H or a combination thereof.

31. The method of claim 23, wherein the RAF mutation comprises S257L/W, S259F, R391S/W, or a combination thereof.

32. The method of claim 23, wherein the one or more alterations comprises NRAS Q61K, KRAS G12C, KRAS G13D, KRAS Q61K, or a combination thereof.

33. The method of any one of claims 1-32, wherein Compound I is:

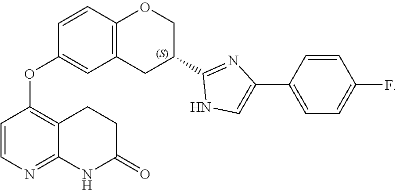

34. The method of claim 33, wherein the compound has an enantiomeric excess of at least 85%, at least 90%, at least 95%, or at least 99%.

35. A method of treating cancer harboring one or more alterations in the mitogen activated protein kinase (MAPK) pathway in a subject, comprising:
detecting the one or more alterations in the MAPK pathway; and then
administering about 5 mg to about 500 mg of Compound I: 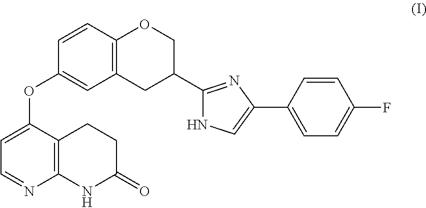
or an equivalent dose of a pharmaceutically acceptable salt, to the subject.

36. The method of claim 35, wherein about 5 mg, about 10 mg, about 20 mg, about 25 mg, about 30 mg, about 40 mg, about 50 mg, about 60 mg, about 75 mg, about 80 mg, about 100 mg, about 150 mg, about 175 mg, about 200 mg, about 225 mg, about 275 mg, about 300 mg, about 325 mg, about 375 mg, about 425 mg, about 475 mg, or about 500 mg of Compound I, or an equivalent dose of a pharmaceutically acceptable salt thereof, is administered.

37. The method of claim 35, wherein about 20 mg to about 500 mg of Compound I, or an equivalent dose of a pharmaceutically acceptable salt thereof, is administered per day.

38. The method of claim 35, wherein about 40 mg to about 500 mg of Compound I, or an equivalent dose of a pharmaceutically acceptable salt thereof, is administered per day.

39. The method of claim 35, wherein about 20-200 mg of Compound I, or an equivalent dose of a pharmaceutically acceptable salt thereof, is administered per day.

40. The method of claim 35, wherein Compound I, or an equivalent dose of a pharmaceutically acceptable salt thereof, is administered once a day or twice a day.

41. The method of claim 40, wherein about 20 mg of Compound I, or an equivalent dose of a pharmaceutically acceptable salt thereof, is administered twice a day.

42. The method of claim 40, wherein about 30 mg of Compound I, or an equivalent dose of a pharmaceutically acceptable salt thereof, is administered twice a day.

43. The method of claim 40, wherein about 50 mg of Compound I, or an equivalent dose of a pharmaceutically acceptable salt thereof, is administered once a day.

44. The method of claim 40, wherein about 50 mg of Compound I, or an equivalent dose of a pharmaceutically acceptable salt thereof, is administered twice a day.

45. The method of claim 40, wherein about 75 mg of Compound I, or an equivalent dose of a pharmaceutically acceptable salt thereof, is administered twice a day.

46. The method of claim 40, wherein about 100 mg of Compound I, or an equivalent dose of a pharmaceutically acceptable salt thereof, is administered once a day.

47. The method of claim 40, wherein about 250 mg of Compound I, or an equivalent dose of a pharmaceutically acceptable salt thereof, is administered twice a day.

48. The method of claim 40, wherein about 500 mg of Compound I, or an equivalent dose of a pharmaceutically acceptable salt thereof, is administered twice a day.

49. The method of claim 35, wherein the one or more alterations is a KRAS mutation, a NRAS mutation, a HRAS mutation, a BRAF mutation, a BRAF fusion, a BRAF translocation, an ARAF mutation, a RAF mutation, an increased receptor tyrosine kinase (RTK) signaling, a RAS gene amplification, a RAF gene amplification or a combination thereof.

50. The method of claim 49, wherein the KRAS mutation is G12C, G12D, G12R, G12V, G12W, G13C, G13D, G13R, G13V, G13W, Q61R, Q61K, Q61L, Q61H, Q61P, A146P, A146T, A146V, A59G, A59T, R68S, H95D, H95Q, H95R, Y96C, or a combination thereof.

51. The method of claim 49, wherein the NRAS mutation comprises Q61R, Q61K, Q61L, Q61H, Q61P, G12S, G12D, G12C, G12V, G12A, G12R, G13V, G13R, G13D, G13C, or a combination thereof.

52. The method of claim 49, wherein the HRAS mutation comprises Q61R, Q61K, Q61L, G12S, G12D, G12C, G12V, G12A, G12R, G13V, G13R, G13D, G13C, or a combination thereof.

53. The method of claim 49, wherein the ARAF mutation comprises G331C, A225V, F351L, P539H, Q347_A348del, N217I, G322S, A451T, R255Gfs(Ter)37, S214(Ter), P8Lfs(Ter)26, G387N/D/R/S, P462L, G377R, or a combination thereof.

54. The method of claim 49, wherein the BRAF mutation comprises Class 1 BRAF mutation, Class 2 BRAF mutation, Class 3 BRAF mutation, or a combination thereof.

55. The method of claim 49, wherein the BRAF mutation comprises Class 2 or 3 BRAF mutation.

56. The method of claim 49, wherein the BRAF mutation comprises V600E, V600M, V600G, V600K, V600R, V600D, G469A, G469V, G469R, K601E, K601N, K601T, L597Q, L597V, G464V, G464E, D594N, D594G, D594A, D594H, G466A, G466V, G466E, N581S, N581I, G469E, S467L, G596D, G596R, F595L, V459L, D287H or a combination thereof.

57. The method of claim 49, wherein the RAF mutation comprises S257L/W, S259F, R391S/W, or a combination thereof.

58. The method of claim 35, wherein the one or more alterations comprises NRAS Q61K, KRAS G12C, KRAS G13D, KRAS Q61K, or a combination thereof.

59. The method of any one of claims 35-58, wherein the cancer is Barret's adenocarcinoma, biliary tract carcinomas, breast cancer, cervical cancer, cholangiocarcinoma, central nervous system tumors, primary CNS tumors, glioblastomas, astrocytomas, glioblastoma multiforme, ependymomas, secondary CNS tumors (metastases to the central nervous system of tumors originating outside of the central nervous system), brain tumors, brain metastases, colorectal cancer, large intestinal colon carcinoma, gastric cancer, carcinoma of the head and neck, squamous cell carcinoma of the head and neck, acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma, erythroleukemia, hepatocellular carcinoma, lung cancer, small cell lung cancer, non-small cell lung cancer, ovarian cancer, endometrial cancer, pancreatic cancer, pituitary adenoma, prostate cancer, renal cancer, melanoma, neurofibromatosis type 1 (NF1), neurofibromatosis type 2 (NF2), esophageal cancer, bladder cancer, mesothelioma, or thyroid cancer.

60. The method of claim 59, wherein the cancer is non-small cell lung cancer (NSCLC), colorectal cancer, melanoma, or pancreatic cancer.

61. The method of any one of claims 35-60, wherein Compound I is:

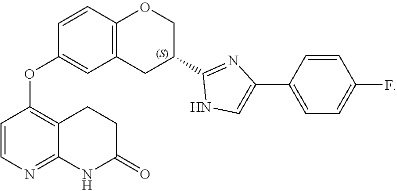

62. The method of claim 61, wherein the compound has an enantiomeric excess of at least 85%, at least 90%, at least 95%, or at least 99%.

63. The method of any one of claims 35-62, wherein the treatment reduces the tumor size compared to the tumor size prior to administration.

64. The method of claim 63, wherein the treatment reduces tumor size by at least about 5%, about 10%, about 20%, about 30%, about 40%, about 50% about 60%, about 70%, about 80%, about 90%, about 95%, or about 100%, compared to the tumor size prior to administration.

65. The method of any one claims 35-64, wherein the treatment increases overall response rate (ORR) compared to the ORR of an otherwise similar patient with cancer that is not treated with Compound I.

66. The method of claim 65, wherein the treatment increases ORR by at least about 5%.

67. The method of claim 66, wherein the treatment increases ORR by 10% to about 100%.

68. The method of any one of claims 35-64, wherein the treatment increases progression free survival (PFS) compared to the PFS of an otherwise similar patient with cancer that is not treated with Compound I.

69. The method of claim 68, wherein the treatment increases PFS by at least about 3 months.

70. The method of claim 69, wherein the treating increases PFS by at least about 6 months, 12 months, at least about 18 months, or at least about 24 months.

71. The method of any one of claims 35-64, wherein the treatment increases overall survival (OS) compared to the OS of an otherwise similar patient with cancer that is not treated with Compound I.

72. The method of claim 71, wherein the treatment increases OS by at least about 3 months.

73. A method of treating cancer expressing one or more ARAF mutants in a patient in need thereof, comprising:
administering about 5 mg to about 500 mg of Compound I: 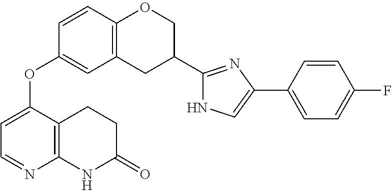
or an equivalent dose of a pharmaceutically acceptable salt thereof, to the subject in need thereof.

74. The method of claim 73, wherein the ARAF mutant comprises a mutation of G331C, A225V, F351L, P539H, Q347_A348del, N217I, G322S, A451T, R255Gfs(Ter)37, S214(Ter), P8Lfs(Ter)26, G387N/D/R/S, P462L, G377R, or a combination thereof.

75. The method of claim 73 or 74, wherein the ARAF mutant comprises a mutation of P462L, G377R, G387D, or a combination thereof.

76. The method of any one of claims 73-75, wherein the Compound I or a pharmaceutically acceptable salt thereof is administered at a dose providing a steady state plasma concentration that is sufficient to inhibit the activity of the ARAF mutant.

77. The method of any one of claims 73-76, wherein about 10 mg to about 300 mg of Compound I, or an equivalent dose of a pharmaceutically acceptable salt thereof, is administered per day.

78. The method of claim 77, wherein about 20 mg to about 200 mg of Compound I, or an equivalent dose of a pharmaceutically acceptable salt thereof, is administered per day.

79. The method of claim 73, wherein about 5 mg, about 20 mg, about 25 mg, about 30 mg, about 40 mg, about 50 mg, about 60 mg, about 75 mg, about 150 mg, about 175 mg, about 200 mg, about 225 mg, about 275 mg, about 300 mg, about 325 mg, about 375 mg, about 425 mg, about 475 mg, or about 500 mg of Compound I, or an equivalent dose of a pharmaceutically acceptable salt thereof, is administered per day.

80. The method of claim 73, wherein Compound I, or an equivalent dose of a pharmaceutically acceptable salt thereof, is administered once a day or twice a day.

81. The method of claim 80, wherein about 20 mg of Compound I, or an equivalent dose of a pharmaceutically acceptable salt thereof, is administered twice a day.

82. The method of claim 80, wherein about 30 mg of Compound I, or an equivalent dose of a pharmaceutically acceptable salt thereof, is administered twice a day.

83. The method of claim 80, wherein about 50 mg of Compound I, or an equivalent dose of a pharmaceutically acceptable salt thereof, is administered once a day.

84. The method of claim 80, wherein about 50 mg of Compound I, or an equivalent dose of a pharmaceutically acceptable salt thereof, is administered twice a day.

85. The method of claim 80, wherein about 75 mg of Compound I, or an equivalent dose of a pharmaceutically acceptable salt thereof, is administered twice a day.

86. The method of claim 80, wherein about 100 mg of Compound I, or an equivalent dose of a pharmaceutically acceptable salt thereof, is administered once a day.

87. The method of claim 80, wherein about 250 mg of Compound I, or an equivalent dose of a pharmaceutically acceptable salt thereof, is administered twice a day.

88. The method of claim 80, wherein about 500 mg of Compound I, or an equivalent dose of a pharmaceutically acceptable salt thereof, is administered twice a day.

89. A method of treating belvarafenib resistant cancer in a patient in need thereof, comprising:
administering about 5 mg to about 500 mg of Compound I 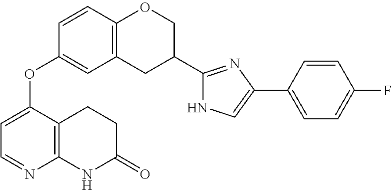
or an equivalent dose of a pharmaceutically acceptable salt thereof to the patient.

90. The method of claim 89, wherein the belvarafenib resistant cancer comprises an ARAF mutant.

91. The method of claim 89 or 90, wherein the ARAF mutant comprises a mutation of P462L, G377R, G387D, or a combination thereof.

92. The method of claim 89, wherein Compound I, or an equivalent dose of a pharmaceutically acceptable salt thereof, is administered once a day or twice a day.

93. The method of claim 92, wherein about 20 mg of Compound I, or an equivalent dose of a pharmaceutically acceptable salt thereof, is administered twice a day.

94. The method of claim 92, wherein about 30 mg of Compound I, or an equivalent dose of a pharmaceutically acceptable salt thereof, is administered twice a day.

95. The method of claim 92, wherein about 50 mg of Compound I, or an equivalent dose of a pharmaceutically acceptable salt thereof, is administered once a day.

96. The method of claim 92, wherein about 50 mg of Compound I, or an equivalent dose of a pharmaceutically acceptable salt thereof, is administered twice a day.

97. The method of claim 92, wherein about 75 mg of Compound I, or an equivalent dose of a pharmaceutically acceptable salt thereof, is administered twice a day.

98. The method of claim 92, wherein about 100 mg of Compound I, or an equivalent dose of a pharmaceutically acceptable salt thereof, is administered once a day.

99. The method of claim 92, wherein about 250 mg of Compound I, or an equivalent dose of a pharmaceutically acceptable salt thereof, is administered twice a day.

100. The method of claim 92, wherein about 500 mg of Compound I, or an equivalent dose of a pharmaceutically acceptable salt thereof, is administered twice a day.
